(19) United States
(12) Patent Application Publication
(10) Pub. No.: US 2026/0015675 A1
(43) Pub. Date: Jan. 15, 2026

(54) KINASE DOMAIN INHIBITORS FOR TREATMENT OF ANGIOSARCOMAS WITH ALTERATION IN KDR GENE

(71) Applicant: Foundation Medicine, Inc., Boston, MA (US)

(72) Inventors: Justin ALLEN, Newton, MA (US); Christopher DARCY, Brookline, MA (US); Alex KESSLER, Goshen, MA (US); Merrida CHILDRESS, Nashville, TN (US)

(73) Assignee: Foundation Medicine, Inc., Boston, MA (US)

(21) Appl. No.: 19/265,865

(22) Filed: Jul. 10, 2025

Related U.S. Application Data
(60) Provisional application No. 63/670,547, filed on Jul. 12, 2024.

(51) Int. Cl. C12Q 1/6886; A61K 31/404; A61K 31/4412; A61K 31/4439; A61K 31/465; A61K 31/47; A61K 31/5025

(52) U.S. Cl. CPC C12Q 1/6886; A61K 31/404; A61K 31/4412; A61K 31/4439; A61K 31/465; A61K 31/47; A61K 31/5025; C12Q 2600/156

(57) ABSTRACT

The present disclosure provides non-frameshift deletions of KDR methods for evaluating, identifying, assessing, and/or treating an individual having a cancer. The present disclosure also provides method of classifying one or more regions of KDR as pathogenic in a preselected group of cancer ontologies.

### CROSS-REFERENCE TO RELATED APPLICATIONS

This application claims the priority benefit of U.S. Provisional Application No. 63/670,547 filed Jul. 12, 2024, the disclosures of which are herein incorporated by reference in their entirety.

### FIELD

The present disclosure relates to non-frameshift deletions in KDR, methods of treating disease such as cancer, with the non-frameshift deletions in KDR and methods of classifying regions of KDR as pathogenic in cancer ontologies.

### BACKGROUND

Cancer represents the phenotypic end-point of multiple genetic lesions that endow cells with a full range of biological properties required for tumorigenesis. Indeed, a hallmark genomic feature of many cancers is the presence of numerous complex chromosome structural aberrations, including translocations, intra-chromosomal inversions, point mutations, deletions, gene copy number changes, gene expression level changes, gene fusions, and germline mutations, among others.

The kinase insert domain receptor (KDR) gene (also known as the Vascular Endothelial Growth Factor Receptor 2 (VEGFR2) gene) is a protein-coding gene responsible for one of the two receptors of the vascular endothelial growth factor (VEGF) which contributes to the growth of endothelial cells. Alterations in the KDR gene have been previously associate with hemangioma and angioma.

However, the pathogenicity of many KDR variants are unknown, thus demonstrating a need to classify KDR variants with current unknown or nonvirulent statuses as virulent/pathogenic in order to be able to predict which patients will be more likely to respond to targeted therapies (e.g., bevacizumab, and kinase domain inhibitors such as pazopanib, sorafenib, sunitinib, and apatinib). Accordingly, there is a need in the art for classification of variants as pathogenic in order to predict response to target therapies and for treatment decisions.

All references cited herein, including patents, patent applications and publications, are hereby incorporated by reference in their entirety. To the extent that any reference incorporated by reference conflicts with the instant disclosure, the instant disclosure shall control.

### BRIEF SUMMARY

Provided herein are methods of identifying an individual having cancer who may benefit from a kinase domain inhibitor and treating individuals with kinase domain inhibitors based on detecting alteration in KDR and labeling the alteration as pathogenic if the alteration is a non-frameshift alteration affecting amino acids 630-753 of KDR. The variant may be labeled as non-pathogenic if the alteration is not a non-frameshift insertion or deletion affecting amino acids 630-753 of KDR.

Also provided herein are methods of classifying regions of KDR as pathogenic in a preselected group cancer ontologies. The methods comprise obtaining knowledge of variants in the KDR gene from individuals with cancer and identifying regions of KDR enriched for mutations in the preselected group of cancer ontologies compared to all cancer ontologies. The preselected cancer ontologies are selected based on expectations that alterations in KDR are driver mutations in the cancer type.

Also provided herein are methods of identifying an individual having cancer who may benefit from a treatment comprising a kinase domain inhibitor, the method comprising: detecting an alteration in kinase insert domain receptor (KDR) in a sample from the individual; labeling the alteration as pathogenic if the alteration comprises a non-frameshift deletion affecting amino acid 630-753 of KDR and/or a non-frameshift insertion affecting amino acids 630-753 of KDR; wherein the labeling of the alteration as pathogenic identifies the individual as an individual who may benefit from treatment comprising the kinase domain inhibitor.

Also provided herein are methods of treating or delaying progression of cancer in an individual having cancer, the method comprising: detecting an alteration in kinase insert domain receptor (KDR) in a sample from the individual; labeling the alteration as pathogenic if the alteration comprises a non-frameshift deletion affecting amino acid 630-753 of KDR and/or a non-frameshift insertion affecting amino acids 630-753 of KDR; administering to the individual a treatment comprising a kinase domain inhibitor if the alteration is labeled as pathogenic.

In some aspects, the methods further comprising obtaining the sample from the individual.

In some aspects, the kinase domain inhibitor is selected from a group consisting of KDR kinase inhibitors include sorafenib, sunitinib, cabozantinib, regorafenib, axitinib, apatinib, and ponatinib.

In some aspects, the alterations labeled as pathogenic are any of the alterations of Table 2.

Also provided herein are methods for classifying one or more regions of kinase insert domain receptor (KDR) as pathogenic in a preselected group of cancer ontologies, comprising: obtaining knowledge of a plurality of variants in KDR that have been detected in samples from a plurality of individuals having cancer; mapping the plurality of variants along the length of KDR by genomic location; identifying one or more regions of KDR with one or more variants from the plurality of variants associated with a cancer from the preselected group of cancer ontologies and without variants from the plurality of variants associated with cancers not in the preselected group of cancer ontologies; and classifying the identified one or more regions of KDR as pathogenic in the preselected group of cancer ontologies.

In some aspects, the cancer is a cancer from a preselected group of cancer ontologies. In some aspects, preselected group of cancer ontologies comprise vascular tumors and melanoma. In some aspects, the vascular tumors comprise angiosarcoma, cardiac sarcoma, hemangioma, and hemangioendothelioma. In some aspects, the preselected group of cancer ontologies comprise breast angiosarcoma, heart sarcoma, kidney sarcoma, liver angiosarcoma, liver hemangioendothelioma, soft tissue angiosarcoma, skin sarcoma, soft tissue angiosarcoma, soft tissue hemangioendothelioma, soft tissue leiomyosarcoma, soft tissue sarcoma, unknown primary sarcoma, head and neck melanoma, skin melanoma, and unknown primary melanoma.

In some aspects, the one or more regions of KDR are within amino acid 630 to amino acid 753 of KDR. In some aspects, one or more regions of KDR comprise amino acid 665 to amino acid 743 of KDR. In some aspects, one or more selected variants in the one or more regions of KDR are classified as pathogenic. In some aspects, the one or more selected variant comprise any of the variants in Table 2.

In some aspects, the plurality of variants comprise non-frameshift alterations. In some aspects, the non-frameshift alterations comprise non-frameshift insertions, non-frameshift deletions, and/or substitutions.

In some aspects, the methods comprise obtaining knowledge of a plurality of variants in KDR that have been detected in samples from a plurality of individuals having cancer comprise detecting the plurality of variants in KDR in the samples. In some aspects, detecting the plurality of variants in KDR are detected in the samples by one or more methods selected from the group consisting of a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), and mass-spectrometric genotyping.

In some aspects, the samples comprise tissue biopsy samples and/or liquid biopsy samples from the plurality of individuals having cancer. In some aspects, the cancer any one of acute myeloid leukemia, bile duct adenocarcinoma, bladder urothelial carcinoma, bone marrow lymphoproliferative disease, bone marrow multiple myeloma, bone marrow myeloproliferative neoplasm, brain anaplastic astrocytoma, brain glioblastoma, brain glioma, breast angiosarcoma, breast cancer, breast carcinoma, breast ductal carcinoma in situ, breast invasive ductal carcinoma, breast invasive lobular carcinoma, cervix adenocarcinoma, cervix squamous cell carcinoma, cholangiocarcinoma, colon adenocarcinoma, colon cancer, duodenum adenocarcinoma, esophagus adenocarcinoma, esophagus carcinoma, esophagus squamous cell carcinoma, eye intraocular melanoma, fallopian tube endometrioid carcinoma, gallbladder adenocarcinoma, gastroesophageal junction adenocarcinoma, head and neck carcinoma, head and neck melanoma, head and neck squamous cell carcinoma, heart sarcoma, kidney clear cell carcinoma, kidney collecting duct carcinoma, kidney renal cell carcinoma, kidney sarcoma, kidney urothelial carcinoma, liver angiosarcoma, liver cholangiocarcinoma, liver hemangioendothelioma, liver intrahepatic cholangiocarcinoma, lung adenocarcinoma, lung adenoid cystic carcinoma, lung cancer, lung large cell neuroendocrine carcinoma, lung non-small cell lung carcinoma, lung sarcomatoid carcinoma, lung small cell undifferentiated carcinoma, lung squamous cell carcinoma, lymph node leukemia lymphocytic acute, ovary endometrioid adenocarcinoma, ovary epithelial carcinoma, ovary germ cell tumor, ovary high grade serous carcinoma, ovary serous carcinoma, pancreas adenosquamous carcinoma, pancreas carcinoma, pancreas ductal adenocarcinoma, pancreas solid pseudopapillary tumor, pancreatobiliary carcinoma, pediatric soft tissue angiosarcoma, prostate acinar adenocarcinoma, prostate cancer, prostate ductal adenocarcinoma, rectum adenocarcinoma, salivary gland carcinoma, salivary gland duct carcinoma, skin basal cell carcinoma, skin melanoma, skin merkel cell carcinoma, skin sarcoma, skin squamous cell carcinoma, soft tissue angiosarcoma, soft tissue clear cell sarcoma, soft tissue ewing sarcoma, soft tissue fibromatosis, soft tissue hemangioendothelioma, soft tissue leiomyosarcoma, soft tissue sarcoma, soft tissue sarcoma undifferentiated, stomach adenocarcinoma, stomach adenocarcinoma diffuse type, stomach adenocarcinoma intestinal type, stomach carcinoma, thymus carcinoma, thyroid carcinoma, unknown primary adenocarcinoma, unknown primary cancer, unknown primary carcinoma, unknown primary flh malignant neoplasm, unknown primary gist, unknown primary malignant neoplasm, unknown primary melanoma, unknown primary sarcoma, unknown primary squamous cell carcinoma, unknown primary urothelial carcinoma, ureter urothelial carcinoma, uterus endometrial adenocarcinoma, uterus endometrial adenocarcinoma endometrioid, uterus endometrial adenocarcinoma mixed histology, and vulva squamous cell carcinoma.

In some aspects, the methods further comprises: providing a plurality of nucleic acid molecules obtained from the sample from the subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; and receiving, using one or more processors, sequence read data for the plurality of sequence reads, wherein the plurality of sequence reads comprise one or more sequence reads comprising an alteration in KDR.

In some aspects, the sample comprises a tissue biopsy sample, a liquid biopsy sample, or a normal control. In some aspects, the sample is a liquid biopsy sample and comprises blood, plasma, cerebrospinal fluid, sputum, stool, urine, or saliva. In some aspects, the sample is a liquid biopsy sample and comprises circulating tumor cells (CTCs). In some aspects, the sample is a liquid biopsy sample and comprises cell-free DNA (cfDNA). In some aspects, the cell-free DNA (cfDNA) or a portion thereof comprises circulating tumor DNA (ctDNA). In some aspects, the plurality of nucleic acid molecules comprises a mixture of tumor nucleic acid molecules and non-tumor nucleic acid molecules. In some aspects, the tumor nucleic acid molecules are derived from a tumor portion of a heterogeneous tissue biopsy sample, and the non-tumor nucleic acid molecules are derived from a normal portion of the heterogeneous tissue biopsy sample. In some aspects, sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

In some aspects, the one or more adapters comprise amplification primers, flow cell adaptor sequences, substrate adapter sequences, or sample index sequences. In some aspects, the captured nucleic acid molecules are captured from the amplified nucleic acid molecules by hybridization to one or more bait molecules. In some aspects, the one or more bait molecules comprise one or more nucleic acid molecules, each comprising a region that is complementary to a region of a captured nucleic acid molecule. In some aspects, amplifying nucleic acid molecules comprises performing a polymerase chain reaction (PCR) amplification technique, a non-PCR amplification technique, or an isothermal amplification technique.

In some aspects, the sequencing comprises use of a massively parallel sequencing (MPS) technique, whole genome sequencing (WGS), whole exome sequencing, targeted sequencing, direct sequencing, or Sanger sequencing technique. In some aspects, the sequencing comprises massively parallel sequencing, and the massively parallel sequencing technique comprises next generation sequencing (NGS). In some aspects, the sequencer comprises a next generation sequencer.

In some aspects, one or more of the plurality of sequencing reads overlap one or more gene loci within one or more subgenomic intervals in the sample. In some aspects, the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci. In some aspects, the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2RIA, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof. In some aspects, the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1B, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.

In some aspects, the methods further comprising generating, by the one or more processors, a report indicating the presence or absence of a KDR alteration labeled as pathogenic.

In some aspects, the methods further comprising transmitting the report to a healthcare provider. In some aspects, the report is transmitted via a computer network or a peer-to-peer connection.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIGS. 1A-1G provide non limiting examples of a summary of frequent genomic alterations in patients with angiosarcoma and hemangioendothelioma. FIG. 1A demonstrates frequently altered genes in all patients with liver hemangioendothelioma. FIG. 1B demonstrates frequently altered genes in all patients with soft tissue hemangioendothelioma. FIG. 1C demonstrates frequently altered genes in pediatric patients with soft tissue hemangioendothelioma. FIG. 1D demonstrates frequently altered genes in all patients with liver angiosarcoma. FIG. 1E demonstrates frequently altered genes in pediatric patients with liver angiosarcoma. FIG. 1F demonstrates frequently altered genes in all patients with angiosarcoma.

FIG. 1G demonstrates frequently altered genes in pediatric patients with angiosarcoma.

FIGS. 2A-2G provide non-limiting examples of histopathology of a liver biopsy. FIG. 2A shows a low power (×20) image of one representative core showing hyalinised fibrotic stroma on the left, lobules of vessels in the middle portion and highly cellular angiosarcoma on the right.

FIG. 2B shows an intermediate power (×200) image of middle portion of core showing lobules of vessels including dilated and smaller ones lined by largely single layer endothelium. Some bile ducts (2 arrowed) were present in the hyalinised fibrotic stroma. FIG. C shows a high power image (×630) of angiosarcoma area with plump and spindled hyperchromatic cells and irregular or slit-like lumina. Three mitosis are highlighted, the upper one in metaphase and the two lower ones in prophase. FIG. 2D shows and Immunostain for glucose transporter 1 (GLUT-1) (×100), in the same image field as shown in FIG. 2B from the middle portion of core, showing diffuse positive staining in the endothelial cells. Note negative reaction of bile ducts in the background. FIG. 2E shows Immunostain for GLUT-1 (×100) in the angiosarcoma are showing diffuscly stained tumor cells. FIG. 2F shows an Immunostain for ERG (×100) in the angiosarcoma, which is a nuclear marker for endothelial cells, showing diffusely stained tumor cells. FIG. 2G shows an Immunostain for KI67 in the Angiosarcoma, showing high proliferative ratio in the tumor (×100).

FIGS. 3A and 3B show a lollypop plot demonstrating the genomic location and density of non-frameshift insertions in all cancer ontologies expected to have pathogenic KDR mutations (FIG. 3A) and in all cancer ontologies (FIG. 3B).

FIGS. 4A and 4B show a lollypop plot demonstrating the genomic location and density of non-frameshift deletions in all cancer ontologies expected to have pathogenic KDR mutations (FIG. 4A) and in all cancer ontologies (FIG. 4B).

FIGS. 5A and 5B show a lollypop plot demonstrating the genomic location and density of non-frameshift insertions and non-frameshift deletions (non-frameshift indels) in all cancer ontologies expected to have pathogenic KDR mutations (FIG. 5A) and in all cancer ontologies (FIG. 5B).

### DETAILED DESCRIPTION

The present disclosure is based, at least in part, on the discovery of alterations of the in kinase insert domain receptor (KDR) gene that are likely to be pathogenic. As more and more targeted therapies are developed, it is important to classify the pathogenicity of mutations on a variant level rather than a gene level. It was discovered that non-frameshift alterations such as non-frameshift alteration, non-frameshift deletions and substitutions between amino acid 630 and 752 of KDR are likely to be pathogenic and thus a cancer with the pathogenic alteration can be treated with a kinase domain inhibitor. Alterations outside of this region may have a reduced likelihood of pathogenicity and thus cancers with the alterations would be less likely to respond to kinase domain inhibitors.

The present disclosure is based, at least in part, on the discovery of an enrichment of KDR alterations between amino acid 630 and 752 of KDR in a subset of cancer ontologies. The cancer ontologies have been linked to pathogenic KDR mutations. Taking advantage of a large dataset of samples, the inventors were able to establish boundaries in the selected ontologies wherein the KDR alterations are expected to be pathogenic.

As used herein, pathogenic alteration refers to an alteration in the DNA sequence that causes or increases risk for cancer. The pathogenic variant may be somatic and/or a germline variant. The pathogenic variant may be a driver mutation for a cancer.

In some aspects, provided herein are methods of treating or delaying progression of cancer. In other aspects, provided herein are methods of identifying one or more treatment options for an individual having cancer. In other aspects, provided herein are methods of selecting treatment for an individual having cancer. In other aspects, provided herein are methods of identifying an individual having cancer who may benefit from a treatment comprising an anti-cancer therapy. In other aspects, provided herein are methods of selecting a therapy for an individual having cancer. In some embodiments, the cancer comprises a non-frameshift insertion or non-frameshift deletion between amino acid 630 and amino acid 752 of KDR. In some embodiments, the methods comprise acquiring knowledge of a non-frameshift insertion or non-frameshift deletion between amino acid 630 and amino acid 752 of KDR in a sample from an individual. In some embodiments, the methods comprise detecting a non-frameshift insertion or non-frameshift deletion between amino acid 630 and amino acid 752 of KDR in a sample from an individual. In some embodiments, the methods comprise generating a report comprising one or more treatment options for the individual. In some embodiments, the methods comprise administering to an individual having cancer an effective amount of a treatment comprising an kinase domain inhibitor. In some embodiments, the cancer is a vascular cancer or a melanoma. In some embodiments, the individual is a human.

Hepatic vascular tumors represent a spectrum of entities that range from benign to malignant histologist, frequently of pediatric onset. The management of these conditions are challenging due to the variation in clinical behavior and frequent lack of pathologic confirmation due to risk of procedural hemorrhage. Symptomatology could be attributed to mass effect, vascular steal, hemorrhage, consumptive coagulopathy, and thromboembolism. Epithelioid hemangioendothelioma (EHE) and angiosarcoma (AS) are the main types of malignant hepatic vascular tumors and may be challenging to distinguish radiographically (i.e., through imaging alone). Kunimoto, et al., ISSVA Classification of Vascular Anomalies and Molecular Biology. 2022. Int J Mol Sci. 23 (4). ctDNA analysis may also allow a more comprehensive profiling of tumors that display intra-tumoral heterogeneity.

Non-invasive diagnostics may provide for earlier diagnosis and treatment of hepatic vascular tumors. Hepatic angiosarcoma is a rare aggressive soft tissue tumor, with only about 200 cases diagnosed annually worldwide. Neshiwat, et al., Hepatic angiosarcoma. 1992. Am J Med. 93 (2): 219-22. Wang, J. and L. T. Sun, Primary hepatic angiosarcoma: A case report. 2022. World J Clin Cases. 10 (31): 11590-11596; Semelka, et al., MRI features of primary rare malignancies of the liver: A report from four university centres. 2018 European Radiology. 28 (4): 1529-1539. MRI of lesion typically shows nodular enhancement that could be challenging to distinguish from hemangiomas. Semelka, et al., MRI features of primary rare malignancies of the liver: A report from four university centres. 2018 European Radiology, 2018. 28 (4): 1529-1539. Indeed, transformation of hemangioma to angiosarcoma is rarely reported. Nathenson, et al., Angiosarcoma arising in a patient with a 10-year-old hemangioma. 2014. Case Rep Oncol Med. 185323. Hepatic angiosarcoma usually carries poor prognosis and has no standardized treatment guidelines due to its rarity and aggressive nature. Antonescu, et al., KDR activating mutations in human angiosarcomas are sensitive to specific kinase inhibitors. 2009. Cancer Res, 69 (18): 7175-9; Chaudhary, et al., Primary hepatic angiosarcoma. 2015. European Journal of Surgical Oncology. 41 (9): 1137-1143. KDR, CD31, and TP53 mutations are biomarkers strongly linked to angiosarcomas. Chaudhary, P., et al., Primary hepatic angiosarcoma. 2015. European Journal of Surgical Oncology. 41 (9): 1137-1143; Yang, et al., Apatinib treatment for KIT- and KDR-amplified angiosarcoma: a case report. 2018. BMC Cancer. 18 (1): 618.

Thus, the methods described herein may also be used to classify hepatic vascular tumors based on the presence of pathogenic alterations the KDR gene and classification of the tumors may aid in treatment selection.

### Definitions

Before describing the invention in detail, it is to be understood that this invention is not limited to particular compositions or biological systems. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “or” is used herein to mean, and is used interchangeably with, the term “and/or”, unless context clearly indicates otherwise.

The terms “about” or “approximately” as used herein refer to the usual error range for the respective value readily known to the skilled person in this technical field, for example, an acceptable degree of error or deviation for the quantity measured given the nature or precision of the measurements. Reference to “about” or “approximately” a value or parameter herein includes (and describes) embodiments that are directed to that value or parameter per se.

The term “isolated” in the context of a nucleic acid molecule or a polypeptide refers to a nucleic acid molecule or polypeptide being separated from other nucleic acid molecules or polypeptides that are present in the natural source of the nucleic acid molecule or polypeptide. In some certain embodiments, the isolated nucleic acid molecule or polypeptide is free of or substantially free of other cellular material or culture medium when produced by recombinant techniques, or free of or substantially free of chemical precursors or other chemicals when chemically synthesized.

As used herein, the term “configured to hybridize to” indicates that a nucleic acid molecule has a nucleotide sequence with sufficient length and sequence complementarity to the nucleotide sequence of a target nucleic acid to allow the nucleic acid molecule to hybridize to the target nucleic acid, e.g., with a Tₘ of at least 65° C. in an aqueous solution of 1×SCC (150 mM sodium chloride and 15 mM trisodium citrate) and 0.1% SDS. Other hybridization conditions may be used when hybridizing a nucleic acid molecule to a target nucleic acid molecule, for example in the context of a described method.

“Percent (%) sequence identity” with respect to a reference polypeptide or polynucleotide sequence is defined as the percentage of amino acid residues or nucleotides in a sequence that are identical to the amino acid residues or nucleotides in the reference polypeptide or polynucleotide sequence, after aligning the sequences and introducing gaps, if necessary, to achieve the maximum percent sequence identity, and not considering any conservative substitutions as part of the sequence identity.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “effective amount” or a “therapeutically effective amount” of an agent, e.g., an anti-cancer agent, or a pharmaceutical formulation, refers to an amount effective, at dosages and for periods of time necessary, to achieve the desired therapeutic or prophylactic result, e.g., in the treatment or management of a cancer, for example, delaying or minimizing one or more symptoms associated with the cancer. In some embodiments, an effective amount or a therapeutically effective amount of an agent refers to an amount of the agent at dosages and for periods of time necessary, alone or in combination with other therapeutic agents, which provides a therapeutic or prophylactic benefit in the treatment or management of a disease such as a cancer. In some embodiments, an effective amount or a therapeutically effective amount of an agent enhances the therapeutic or prophylactic efficacy of another therapeutic agent or another therapeutic modality.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, delaying progression of disease, alleviation of symptoms, diminishment of any direct or indirect pathological consequences of the disease, decreasing the rate of disease progression, amelioration or palliation of the disease state, and remission or improved prognosis. In some embodiments, the terms “treatment,” “treat,” or “treating” include preventing a disease, such as cancer, e.g., before an individual begins to suffer from a cancer or from re-growth or recurrence of the cancer. In some embodiments, the terms “treatment,” “treat,” or “treating” include inhibiting or reducing the severity of a disease such as a cancer.

“Likely to” or “increased likelihood,” as used herein, refer to an increased probability that an event, item, object, thing or person will occur. Thus, in one example, an individual that is likely to respond to treatment with an anti-cancer therapy, e.g., an anti-cancer therapy provided herein, alone or in combination, has an increased probability of responding to treatment with the anti-cancer therapy alone or in combination, relative to a reference individual or group of individuals. “Unlikely to” refers to a decreased probability that an event, item, object, thing or person will occur relative to a reference individual or group of individuals. Thus, an individual that is unlikely to respond to treatment with an anti-cancer therapy, e.g., an anti-cancer therapy provided herein, alone or in combination, has a decreased probability of responding to treatment with the anti-cancer therapy, alone or in combination, relative to a reference individual or group of individuals.

“Sample,” as used herein, refers to a biological sample obtained or derived from a source of interest, as described herein.

### Detecting and Labeling Non-Frameshift Alterations of KDR as Pathogenic

The methods of the current application relate to alterations/variants in the KDR gene. KDR is a type IV receptor tyrosine kinase. KDR is also known as vascular endothelial growth factor receptor 2 (VEGFR-2). In some embodiments, the KDR gene and/or the location of a KDR alteration is defined according to transcript NM_002253.

The methods described herein comprise detecting an alteration in KDR in a sample from an individual. The sample may be tissue sample or a liquid biopsy sample. The liquid biopsy sample may comprise cfDNA. The cfDNA may comprise ctDNA. In some embodiments, the sample comprises tumor derived nucleic acids (e.g., DNA).

The individual may have or be suspected of having cancer. In some embodiments, the cancer is a vascular cancer or a melanoma. In some embodiments, the cancer is breast angiosarcoma, heart sarcoma, kidney sarcoma, liver angiosarcoma, liver hemangioendothelioma, soft tissue angiosarcoma, skin sarcoma, soft tissue angiosarcoma, soft tissue hemangioendothelioma, soft tissue leiomyosarcoma, soft tissue sarcoma, unknown primary sarcoma, head and neck melanoma, skin melanoma, and unknown primary melanoma.

Detecting an alteration may comprise detecting a variant of a plurality of variants in KDR. In some embodiments, the KDR alteration is detected using any suitable method known in the art or described herein. In some embodiments, the sample is a sample described herein. In some embodiments, the sample comprises cells from the cancer or is obtained from cells from the cancer. The alteration may be detected in a sample from the individual using a method selected from nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), and mass-spectrometric genotyping.

Detecting an alteration may comprise detecting a KDR alteration in a plurality of sequence reads generated from a sample from the individual. In some embodiments, the methods comprise providing a plurality of nucleic acid molecules obtained from the sample from the subject ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; and receiving, using one or more processors, sequence read data for the plurality of sequence reads, wherein the plurality of sequence reads comprise one or more sequence reads comprising an alteration in KDR.

The methods described herein comprise labeling the alteration as pathogenic if the alteration comprises a non-frameshift alteration (e.g., insertion or deletion) affecting amino acid 630-753 of KDR and/or non-frameshift insertion affecting amino acid 630-753 of KDR.

The methods of the current application relate to non-frameshift alterations in KDR. Non-frameshift alterations and/or non-frameshift variants refer to genomic alterations leading to changes in the sequence of an amino acid produced from the DNA but that do not result in a shift in the reading frame use for translation of the amino acid. The non-frameshift alterations may result in amino acid substitutions and may impact the function of the resulting protein. In some embodiments, the non-frameshift alterations result in loss of function of the KDR protein.

The KDR non-frameshift alterations of the present application comprise non-frameshift insertions, non-frameshift deletions, and substitutions. In some embodiments, the substitutions are nonsynonymous substitutions resulting in changes in amino acid sequence.

In some embodiments, the KDR variants are germline variants. In some embodiments, the KDR variants are somatic variants. In some embodiments, somatic KDR variants are detected in samples comprising nucleic acids from a cancer.

In some embodiments, the non-frameshift alterations in KDR are pathogenic variants for one or more cancer ontologies, such as the pre-selected cancer ontologies described herein. The methods may relate to classifying non-frameshift alterations in KDR as likely pathogenic or less likely to be pathogenic in one or more cancer ontologies. The non-frameshift KDR alterations of the present application may be labeled as pathogenic based on the location of the non-frameshift alteration in KDR.

In some embodiments, an alteration in KDR may be labeled as pathogenic if the alteration comprises a non-frameshift deletion or non-frameshift insertion affecting amino acids 630-753 of KDR. In some embodiments, an alteration in KDR may be labeled as less likely to by pathogenic if the alteration comprises a non-frameshift deletion or non-frameshift insertion outside of the region comprising amino acid 630-753 of KDR.

In some embodiments, an alteration in KDR may be labeled as pathogenic if the alteration comprises a non-frameshift deletion or non-frameshift insertion affecting amino acids 630-753 of KDR and was identified in a cancer ontology associated with pathogenic KDR alterations. The cancer ontology may be a vascular cancer or a melanoma. The cancer ontology may be a cancer ontology on a preselected group of cancer ontologies described herein. In some embodiments, an alteration in KDR may be identified as less likely to by pathogenic if the alteration comprises a non-frameshift deletion or non-frameshift insertion outside of the region comprising amino acid 630-753 of KDR or if the alteration was identified in a cancer ontology less likely to be associated with pathogenic KDR alterations. Such cancer ontologies may include any cancer ontology other than vascular cancers and melanomas. In some embodiments, the pathogenic KDR alteration may be any variant in Table 2.

### Methods Selecting or Identifying a Treatment

In some embodiments, the methods described herein can be used for identifying an individual with cancer who may benefit from a treatment comprising a kinase domain inhibitor.

In some aspects, provided herein are methods of identifying an individual having cancer, e.g., a cancer provided herein, who may benefit from a treatment comprising a kinase domain inhibitor. In some embodiments, the methods comprise detecting an alteration in KDR in a sample from the individual. In some embodiments, the methods comprise detecting the alteration in KDR in a nucleic acid molecule or polypeptide in a sample obtained from the individual. The alteration may be labeled as pathogenic if the alteration comprises a non-frameshift deletion or non-frameshift insertion affecting amino acid 830-753 of KDR.

Also provided herein are methods of identifying or selecting a treatment, a therapy, or one or more treatment options for an individual having a cancer, such as a cancer described herein. In some embodiments, the cancer comprises an alteration in KDR, such as a alteration in KDR labeled as pathogenic according to the methods described herein. The alteration may be labeled as pathogenic if the alteration comprises a non-frameshift deletion or non-frameshift insertion affecting amino acid 830-753 of KDR. In some embodiments, the therapy or treatment comprises a kinase domain inhibitor.

### Methods of Treatment

Also provided herein are methods of treating or delaying progression of a cancer in an individual, such as a cancer provided herein. In some embodiments, the individual has a cancer comprising KDR alteration, such as a KDR alteration labeled as pathogenic according to the methods described herein. The alteration may be labeled as pathogenic if the alteration comprises a non-frameshift deletion or non-frameshift insertion affecting amino acid 830-753 of KDR.

some embodiments, the methods of treating or delaying progression of a cancer of the disclosure in an individual, e.g., comprising a pathogenic KDR alteration, comprise administering to the individual a therapeutically effective amount of a kinase domain inhibitor. In some embodiments, the methods of treating or delaying progression of a cancer of the disclosure in an individual, comprise administering to the individual an effective amount of a kinase domain inhibitor, responsive to knowledge of the presence of the labeling of a KDR alteration detected in a sample from the individual as pathogenic. In some embodiments, the sample is a sample described herein. In some embodiments, the sample comprises cells from the cancer or is obtained from cells from the cancer.

### Reporting

In some embodiments, the methods provided herein comprise generating a report, and/or providing a report to party. In some embodiments, the methods comprise transmitting the report to a healthcare provider. In some embodiments, the report is transmitted via a computer network or a peer-to-peer connection.

In some embodiments, a report according to the present disclosure comprises information about one or more of: an alterations in KDR, such as a non-frameshift insertion or non-frameshift deletion. In some embodiments, the alteration in KDR may be detected in nucleic acid molecules or polypeptides in a sample from an individual. The individual may have or be suspected for having cancer, such as vascular cancer or melanoma. The report may comprise information about a treatment comprising a kinase domain inhibitor if a KDR alteration labeled as pathogenic is identified as in a sample from the individual

In some embodiments, a report according to the present disclosure comprises information about the presence or absence of a KDR alteration of the disclosure in a sample obtained from an individual, such as an individual having a cancer, e.g., a cancer provided herein. In one embodiment, a report according to the present disclosure indicates that a KDR alteration of the disclosure (e.g., alteration labeled as pathogenic) is present in a sample obtained from the individual. In one embodiment, a report according to the present disclosure indicates that a KDR alteration of the disclosure (e.g., alteration labeled as pathogenic) is not present in a sample obtained from the individual. In some embodiments, the report comprises an identifier for the individual from which the sample was obtained.

In some embodiments, the report includes information on the role of an alteration in KDR such as a non-frameshift insertion or deletion of the disclosure (e.g., alteration labeled as pathogenic), or its wild type counterparts, in cancer. Such information can include one or more of: information on prognosis of a cancer, such as a cancer provided herein, e.g., comprising a KDR alteration (e.g., alteration labeled as pathogenic) described herein; information on resistance of a cancer, such as a cancer provided herein, e.g., comprising a KDR alteration described herein, to one or more treatments; information on potential or suggested therapeutic options (e.g., such as a kinase domain inhibitor provided herein, or a treatment selected or identified according to the methods provided herein); or information on therapeutic options that should be avoided. In some embodiments, the report includes information on the likely effectiveness, acceptability, and/or advisability of applying a therapeutic option (e.g., such as a kinase domain inhibitor provided herein, or a treatment selected or identified according to the methods provided herein) to an individual having a cancer, such as a cancer provided herein. In some embodiments, the cancer is a vascular cancer or melanoma. In some embodiments, the report includes information or a recommendation on the administration of a treatment (e.g., a kinase domain inhibitor, or a treatment selected or identified according to the methods provided herein). In some embodiments, the information or recommendation includes the dosage of the treatment and/or a treatment regimen (e.g., in combination with other treatments, such as a second therapeutic agent). In some embodiments, the report comprises information or a recommendation for at least one, at least two, at least three, at least four, at least five, at least six, at least seven, at least eight, at least nine, at least ten, or more treatments.

Also provided herein are methods of generating a report according to the present disclosure. In some embodiments, a report according to the present disclosure is generated by a method comprising one or more of the following steps: obtaining a sample, such as a sample described herein, from an individual, e.g., an individual having a cancer, such as a cancer provided herein; detecting a KDR alteration of the disclosure in the sample (e.g., a KDR alteration labeled as pathogenic); and generating a report. In some embodiments, a report generated according to the methods provided herein comprises one or more of: information about the presence or absence of a KDR alteration in the sample; an identifier for the individual from which the sample was obtained; information on the role of a KDR alteration (e.g., alteration labeled as pathogenic), or its wild type counterparts, in cancer; information on prognosis, resistance, or potential or suggested therapeutic options (such as a kinase domain inhibitor provided herein, or a treatment selected or identified according to the methods provided herein); information on the likely effectiveness, acceptability, or the advisability of applying a therapeutic option (such as a kinase domain inhibitor provided herein, or a treatment selected or identified according to the methods provided herein) to the individual; a recommendation or information on the administration of a treatment (such as a kinase domain inhibitor provided herein, or a treatment selected or identified according to the methods provided herein); or a recommendation or information on the dosage or treatment regimen of a treatment (such as a kinase domain inhibitor provided herein, or a treatment selected or identified according to the methods provided herein), e.g., in combination with other treatments (e.g., a second therapeutic agent). In some embodiments, the report generated is a personalized cancer report.

A report according to the present disclosure may be in an electronic, web-based, or paper form. The report may be provided to an individual or a patient (e.g., an individual or a patient with a cancer, such as a cancer provided herein, or to an individual or entity other than the individual or patient (e.g., other than the individual or patient with the cancer), such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from detecting a KDR alteration of the disclosure in a sample obtained from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from acquiring knowledge of the presence of a KDR alteration of the disclosure in a sample obtained from an individual (e.g., an individual having a vascular cancer or melanoma).

The method steps of the methods described herein are intended to include any suitable method of causing one or more other parties or entities to perform the steps, unless a different meaning is expressly provided or otherwise clear from the context. Such parties or entities need not be under the direction or control of any other party or entity, and need not be located within a particular jurisdiction. Thus, for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

### Methods Classifying Pathogenic Regions of KDR

Provided herein are methods of for classifying one or more regions of KDR as pathogenic in a preselected group of cancer ontologies (e.g., cancer types). The methods can be used for classifying variants in the one or more regions of KDR as pathogenic and for informing treatment decisions in individuals with a cancer from the preselected group of cancer ontologies. The methods comprise obtaining knowledge of a plurality of variant in KDR that have been detected in samples from a plurality of individuals having cancer, mapping the plurality of variants along the length of the KDR by genomic location, identifying one or more regions of KDR with one or more variants from the plurality of variants associated with a cancer from the preselected group of cancers and without variants from the plurality of variants associated with cancers not in the preselected group of cancers and classifying the identified one or more regions of KDR as pathogenic in the preselected group of cancers.

The methods comprise obtaining knowledge of a plurality of variants in KDR that have been detected in samples from a plurality of individuals having cancer. In some embodiments, the samples from the plurality of individuals with cancer comprise tissue biopsy and/or liquid biopsy samples from the plurality of individuals having cancer. In some embodiments, the methods comprise detecting the plurality of variants in KDR in samples from a plurality of individuals having cancer. Methods described herein and known in the art may be used to detect KDR variants in the samples. In some embodiments, the plurality of variants in KDR are detected in the samples by one or more methods selected from the group consisting of a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), and mass-spectrometric genotyping.

In some embodiments, the plurality of variants in KDR comprise KDR alterations. In some embodiments, the alterations in KDR are non-frameshift alterations. In some embodiments, non-frameshift alterations comprise non-frameshift insertions, non-frameshift deletions, and/or substitutions. In some embodiments, the non-frameshift alterations are non-frameshift insertion, non-frameshift deletions. In some embodiments, the non-frameshift alterations are substitutions.

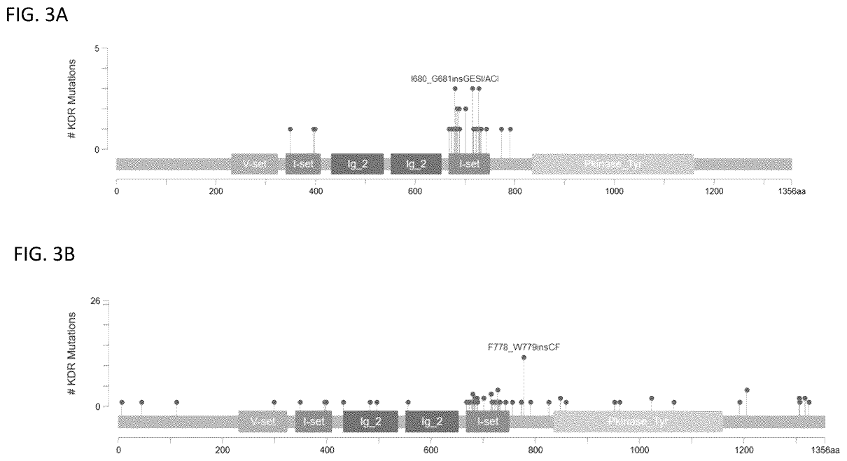

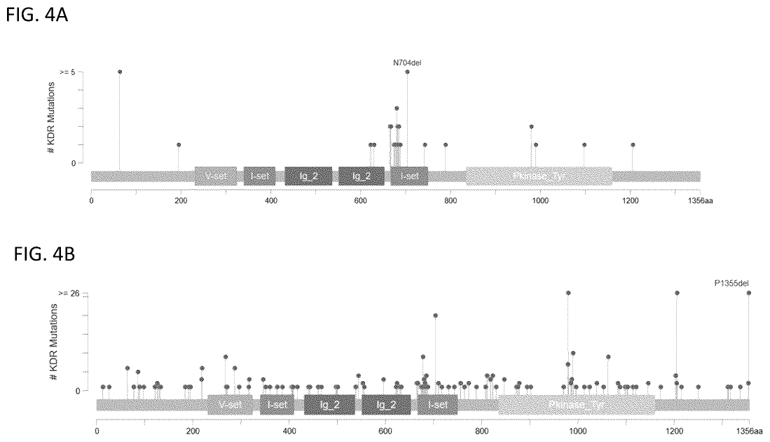

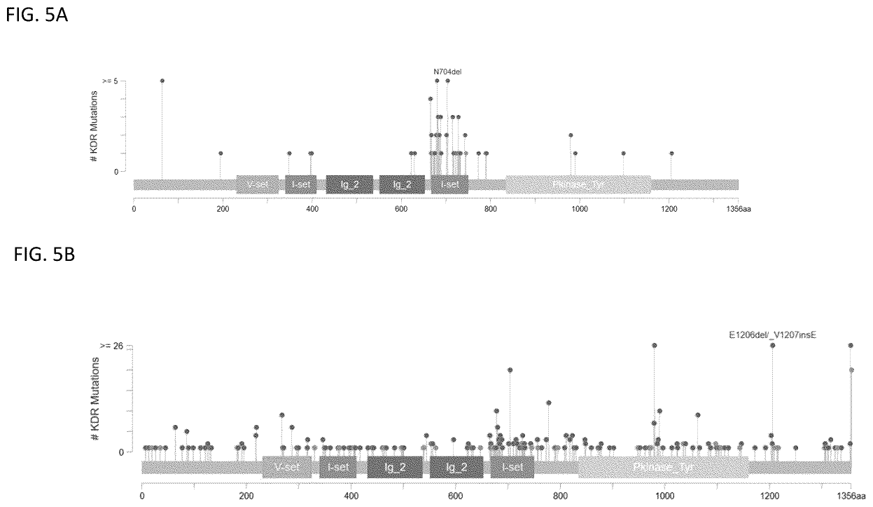

The methods comprise mapping the plurality of variants along the length of KDR by genomic location. In some embodiments, the plurality of variants are mapped along the length of KDR by the amino acid position that would be affected upon translation of the KDR protein. In some embodiments, the plurality of variants are mapped based on exon number. In some embodiments, mapping the plurality of variants comprises annotating a genomic region comprising the KDR gene with the plurality of variants. In a non-limiting examples, mapping the plurality of variants comprises constructing a lollypop plot, such as those shown in FIGS. 3A, 3B, 4A, 4B, 5A, and 5B. The lollypop plots provide a visual representation of where the plurality of variants are located along the gene. In some embodiments, the number of unique KDR variants at a genomic location is annotated.

The methods comprise identifying one or more regions of KDR with one or more variants from the plurality of variants associated with a cancer from a preselected group of cancer ontologies and without variants from the plurality of variants associated with cancers not in the preselected group of cancer ontologies. In some embodiments, the methods comprise selected the preselected group of cancer ontologies. The preselected group of cancer ontologies may be selected based on the association of the cancer ontology with pathogenic KDR alterations.

In some embodiments, the preselected group of cancer ontologies (e.g., cancer types) comprise cancers that associated with KDR alterations or that respond to kinase domain inhibitors. KDR alterations in vascular tumors such as angiosarcoma, have been shown to be pathogenic. In some embodiments, the preselected group of cancers comprise vascular tumors. In some embodiments, the vascular tumors comprise angiosarcoma, cardiac sarcoma, hemangioma, and hemangioendothelioma. In some embodiments, the preselected group of cancer ontologies comprise breast angiosarcoma, heart sarcoma, kidney sarcoma, liver angiosarcoma, liver hemangioendothelioma, soft tissue angiosarcoma, skin sarcoma, soft tissue angiosarcoma, soft tissue hemangioendothelioma, soft tissue leiomyosarcoma, soft tissue sarcoma, and unknown primary sarcoma.

In some embodiments, the preselected group of cancer ontologies (e.g., cancer types) comprise melanomas. In some embodiments, the melanoma is head and neck melanoma, skin melanoma, and unknown primary melanoma.

In some embodiments, the preselected group of cancer ontologies (e.g., cancer types) comprise vascular tumors and melanoma. In some embodiments, the vascular tumors comprise vascular tumors comprise angiosarcoma, cardiac sarcoma, hemangioma, and hemangioendothelioma. In some embodiments, the preselected group of cancer ontologies comprise breast angiosarcoma, heart sarcoma, kidney sarcoma, liver angiosarcoma, liver hemangioendothelioma, soft tissue angiosarcoma, skin sarcoma, soft tissue angiosarcoma, soft tissue hemangioendothelioma, soft tissue leiomyosarcoma, soft tissue sarcoma, unknown primary sarcoma, head and neck melanoma, skin melanoma, and unknown primary melanoma.

In some embodiments, identifying one or more regions of KDR with one or more variants from the plurality of variants associated with a cancer from the preselected group of cancer ontologies and without variants from the plurality of variants associated with cancers not in the preselected group of cancer ontologies comprises visual inspection of where the KDR alterations map by genomic position. In some embodiments, the regions are defined based on identifying breakpoints between KDR variants associated with cancers from the preselected group of cancer ontologies and KDR variants association with cancers not in the preselected group of cancer ontologies. In some embodiments, the methods comprise identifying regions enriched for KDR variants associated with cancers from the preselected group of cancer ontologies. In some embodiments, the one or more regions are about 1.5 times, about 2.0 times, about 2.5 times or more than about 3 times enriched for KDR variation associated with cancers from the preselected group of cancer ontologies.

The methods comprise classifying the identified one or more regions of KDR as pathogenic in the preselected group of cancer ontologies. In some embodiments, the identified one or more regions comprise one or more regions of KDR between amino acid 640 to amino acid 753 of KDR. In some embodiments, the identified one or more regions comprise one or more regions of KDR between amino acid 665 and amino acid 743 of KDR.

In some embodiments, KDR variants within the one or more pathogenic regions of KDR are classified as pathogenic variants. In some embodiments, the one or more regions of KDR comprise amino acid 640 to amino acid 753 of KDR. In some embodiments, one or more selected variants in the one or more regions of KDR are classified as pathogenic. In some embodiments, the one or more selected variants are non-frameshift alterations between amino acid 640 and amino acid 753 of KDR. In some embodiments, non-frameshift alterations comprise non-frameshift insertions, non-frameshift deletions, and/or substitutions. In some embodiments, the non-frameshift alterations are non-frameshift insertion, non-frameshift deletions. In some embodiments, the non-frameshift alterations are substitutions. In some embodiments, the one or more selected variations comprise any of the variants in Table 2.

In some embodiments, KDR variants within the one or more pathogenic regions of KDR are classified as pathogenic variants. In some embodiments, the one or more regions of KDR comprise amino acid 665 and amino acid 743 of KDR. In some embodiments, one or more selected variants in the one or more regions of KDR are classified as pathogenic. In some embodiments, the one or more selected variants are non-frameshift alterations between amino acid 665 and amino acid 743 of KDR. In some embodiments, non-frameshift alterations comprise non-frameshift insertions, non-frameshift deletions, and/or substitutions. In some embodiments, the non-frameshift alterations are non-frameshift insertion, non-frameshift deletions. In some embodiments, the non-frameshift alterations are substitutions. In some embodiments, the one or more selected variations comprise any of the variants in Table 2 that are between amino acid 665 and amino acid 743 of KDR.

As described herein, labeling a region of KDR and or KDR alterations as pathogenic in a preselected group of cancer ontologies informs treatment selection and treatment of individuals with a cancer of the preselected group of cancers.

### Methods of Detecting an Alteration KDR

In some embodiments, the methods disclosed herein comprise detecting an alteration in KDR in a sample from the individual. In some embodiments, the method comprise obtaining knowledge of a plurality of variants in KDR that have been detected in samples from individual having cancer. In some embodiments, the KDR alteration among nucleic acid molecules isolated from the sample of the disclosure may be detected using an in situ hybridization method, such as a fluorescence in situ hybridization (FISH) method.

In some embodiments, FISH analysis is used to identify an RNA molecule comprising a KDR alteration described herein. Methods for performing FISH are known in the art and can be used in nearly any type of tissue. In FISH analysis, nucleic acid probes which are detectably labeled, e.g., fluorescently labeled, are allowed to bind to specific regions of DNA, e.g., a chromosome, or an RNA, e.g., an mRNA, and then examined, e.g., through a microscope. Sec, for example, U.S. Pat. No. 5,776,688. DNA or RNA molecules are first fixed onto a slide, the labeled probe is then hybridized to the DNA or RNA molecules, and then visualization is achieved, e.g., using enzyme-linked label-based detection methods known in the art. Generally, the resolution of FISH analysis is on the order of detection of 60 to 100000 nucleotides, e.g., 60 base pairs (bp) up to 100 kilobase pairs of DNA. Nucleic acid probes used in FISH analysis comprise single stranded nucleic acids. Such probes are typically at least about 50 nucleotides in length. In some embodiments, probes comprise about 100 to about 500 nucleotides. Probes that hybridize with centromeric DNA and locus-specific DNA or RNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA or other sources of nucleic acids through standard techniques. Examples of probes, labeling and hybridization methods are known in the art. In some embodiments, FISH probes designed to hybridize to KDR.

Several variations of FISH methods are known in the art and are suitable for use according to the methods of the disclosure, including single-molecule RNA FISH, Fiber FISH, Q-FISH, Flow-FISH, MA-FISH, break-away FISH, hybrid fusion-FISH, and multi-fluor FISH or mFISH.

Also provided herein are probes, e.g., nucleic acid molecules, suitable for the detection of a KDR alteration in a nucleic acid molecule provided herein. In some embodiments, a probe provided herein comprises a nucleic acid sequence configured to hybridize to a target nucleic acid molecule comprising a KDR nucleic acid molecule provided herein, or a fragment or portion thereof. In some embodiments, the probe comprises a nucleic acid sequence configured to hybridize to a KDR nucleic acid molecule, or a fragment or portion thereof, of the target nucleic acid molecule. In some embodiments, the probe comprises a nucleic acid sequence configured to hybridize to a fragment or portion of the KDR nucleic acid molecule nucleic acid molecule of the target nucleic acid molecule. In some embodiments, the fragment or portion comprises between about 5 and about 25 nucleotides, between about 5 and about 300 nucleotides, between about 100 and about 300 nucleotides, between about 130 and about 230 nucleotides, or between about 150 and about 200 nucleotides.

In some embodiments, the probe comprises a nucleic acid molecule which is a DNA, RNA, or a DNA/RNA molecule. In some embodiments, the probe comprises a nucleic acid molecule comprising any of between about 10 and about 20 nucleotides, between about 12 and about 20 nucleotides, between about 10 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the probe comprises a nucleic acid molecule comprising any of 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides. In some embodiments, the probe comprises a nucleic acid molecule comprising any of between about 40 nucleotides and about 50 nucleotides, about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the probe comprises a nucleic acid molecule comprising between about 12 and about 20 nucleotides.

In some embodiments, a probe provided herein comprises a DNA, RNA, or a DNA/RNA molecule. In some embodiments, a probe provided herein includes a label or a tag. In some embodiments, the label or tag is a radiolabel (e.g., a radioisotope), a fluorescent label (e.g., a fluorescent compound), an enzymatic label, an enzyme co-factor, a sequence tag, biotin, or another ligand. In some embodiments, a probe provided herein includes a detection reagent such as a fluorescent marker. In some embodiments, a probe provided herein includes (e.g., is conjugated to) an affinity tag, e.g., that allows capture and isolation of a hybrid formed by a probe and a nucleic acid hybridized to the probe. In some embodiments, the affinity tag is an antibody, an antibody fragment, biotin, or any other suitable affinity tag or reagent known in the art. In some embodiments, a probe is suitable for solution phase hybridization.

In some embodiments, probes provided herein may be used according to the methods of detection of a KDR alteration within nucleic acid molecules provided herein. For example, a probe provided herein may be used for detecting a KDR alteration provided herein in a sample, e.g., a sample obtained from an individual.

In some embodiments, one or more probes provided herein are suitable for use in in situ hybridization methods, e.g., as described above, such as FISH.

In some embodiments, probes, such as probes for use in the FISH methods described herein, are labeled such that a chromosomal region or a region on an RNA to which the probes hybridize can be detected. Probes typically are directly labeled with a fluorophore, allowing the probe to be visualized without a secondary detection molecule. Probes can also be labeled by nick translation, random primer labeling or PCR labeling. Labeling may be accomplished using fluorescent (direct)- or haptene (indirect)-labeled nucleotides. Representative, non-limiting examples of labels include: AMCA-6-dUTP, Cascade Blue-4-dUTP, Fluorescein-12-dUTP, Rhodamine-6-dUTP, TexasRed-6-dUTP, Cy3-6-dUTP, Cy5-dUTP, Biotin (BIO)-11-dUTP, Digoxygenin (DIG)-11-dUTP and Dinitrophenyl (DNP)-11-dUTP. Probes can also be indirectly labeled with biotin or digoxygenin or labeled with radioactive isotopes such as 32P and 3H, and secondary detection molecules are used, or further processing is performed, to visualize the probes. For example, a probe labeled with biotin can be detected by avidin conjugated to a detectable marker, e.g., avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase. Probes can also be prepared such that a fluorescent or other label is added after hybridization of the probe to its target to detect that the probe hybridized to the target. For example, probes can be used that have antigenic molecules incorporated into the nucleotide sequence. After hybridization, these antigenic molecules are detected, for example, using specific antibodies reactive with the antigenic molecules. Such antibodies can, for example, themselves incorporate a fluorochrome, or can be detected using a second antibody with a bound fluorochrome. For fluorescent probes, e.g., used in FISH techniques, fluorescence can be viewed with a fluorescence microscope equipped with an appropriate filter for each fluorophore, or by using dual or triple band-pass filter sets to observe multiple fluorophores. Alternatively, techniques such as flow cytometry can be used to examine the hybridization pattern of the chromosomal probes.

### Array-Based Methods

In some embodiments, the KDR alterations among nucleic acid molecules isolated from the sample of the disclosure may be detected using an array-based method, such as array-based comparative genomic hybridization (CGH) methods. In array-based CGH methods, a first sample of nucleic acids (e.g., from a sample, such as from a tumor) is labeled with a first label, while a second sample of nucleic acids (e.g., a control, such as from a healthy cell/tissue) is labeled with a second label. In some embodiments, equal quantities of the two samples are mixed and co-hybridized to a DNA microarray of several thousand evenly spaced cloned DNA fragments or oligonucleotides, which have been spotted in triplicate on the array. After hybridization, digital imaging systems are used to capture and quantify the relative fluorescence intensities of each of the hybridized fluorophores. In some embodiments, Array-based CGH can also be performed with single-color labeling. In single color CGH, a control (e.g., control nucleic acid sample, such as from a healthy cell/tissue) is labeled and hybridized to one array and absolute signals are read, and a test sample (e.g., a nucleic acid sample obtained from an individual or from a tumor) is labeled and hybridized to a second array (with identical content) and absolute signals are read.

Provided herein are baits suitable for the detection of KDR alterations of the disclosure. In some instances, the baits are suitable to detect a KDR alteration. In some embodiments, the bait comprises a capture nucleic acid molecule configured to hybridize to a target nucleic acid molecule comprising a KDR nucleic acid molecule provided herein, or a fragment or portion thereof. In some embodiments, the bait comprises a capture nucleic acid molecule configured to hybridize to a target nucleic acid molecule comprising a KDR alteration in a nucleic acid molecule provided herein, or a fragment or portion thereof.

In some embodiments, the capture nucleic acid molecule is configured to hybridize to a fragment of the KDR nucleic acid molecule. In some embodiments, the fragment comprises (or is) between about 5 and about 25 nucleotides, between about 5 and about 300 nucleotides, between about 100 and about 300 nucleotides, between about 130 and about 230 nucleotides, or between about 150 and about 200 nucleotides. In some embodiments, the capture nucleic acid molecule is between about 5 and about 25 nucleotides, between about 5 and about 300 nucleotides, between about 100 and about 300 nucleotides, between about 130 and about 230 nucleotides, or between about 150 and about 200 nucleotides. In some embodiments, the fragment comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length. In some embodiments, the capture nucleic acid molecule comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length.

In some embodiments, a bait provided herein comprises a DNA, RNA, or a DNA/RNA molecule. In some embodiments, a bait provided herein includes a label or a tag. In some embodiments, the label or tag is a radiolabel, a fluorescent label, an enzymatic label, a sequence tag, biotin, or another ligand. In some embodiments, a bait provided herein includes a detection reagent such as a fluorescent marker. In some embodiments, a bait provided herein includes (e.g., is conjugated to) an affinity tag, e.g., that allows capture and isolation of a hybrid formed by a bait and a nucleic acid hybridized to the bait. In some embodiments, the affinity tag is an antibody, an antibody fragment, biotin, or any other suitable affinity tag or reagent known in the art. In some embodiments, a bait is suitable for solution phase hybridization.

Baits can be produced and used according to methods known in the art, e.g., as described in WO2012092426A1 and/or or in Frampton et al (2013) Nat Biotechnol, 31:1023-1031, incorporated herein by reference. For example, biotinylated baits (e.g., RNA baits) can be produced by obtaining a pool of synthetic long oligonucleotides, originally synthesized on a microarray, and amplifying the oligonucleotides to produce the bait sequences. In some embodiments, the baits are produced by adding an RNA polymerase promoter sequence at one end of the bait sequences, and synthesizing RNA sequences using RNA polymerase. In one embodiment, libraries of synthetic oligodeoxynucleotides can be obtained from commercial suppliers, such as Agilent Technologies, Inc., and amplified using known nucleic acid amplification methods.

In some embodiments, a bait provided herein is between about 100 nucleotides and about 300 nucleotides. In some embodiments, a bait provided herein is between about 130 nucleotides and about 230 nucleotides. In some embodiments, a bait provided herein is between about 150 nucleotides and about 200 nucleotides. In some embodiments, a bait provided herein comprises a target-specific bait sequence (e.g., a capture nucleic acid molecule described herein) and universal tails on each end. In some embodiments, the target-specific sequence, e.g., a capture nucleic acid molecule described herein, is between about 40 nucleotides and about 300 nucleotides. In some embodiments, the target-specific sequence, e.g., a capture nucleic acid molecule described herein, is between about 100 nucleotides and about 200 nucleotides. In some embodiments, the target-specific sequence, e.g., a capture nucleic acid molecule described herein, is between about 120 nucleotides and about 170 nucleotides. In some embodiments, the target-specific sequence, e.g., a capture nucleic acid molecule described herein, is about 150 nucleotides or about 170 nucleotides. In some embodiments, a bait provided herein comprises an oligonucleotide comprising about 200 nucleotides, of which about 150 nucleotides or about 170 nucleotides are target-specific (e.g., a capture nucleic acid molecule described herein), and the other 50 nucleotides or 30 nucleotides (e.g., 25 or 15 nucleotides on each end of the bait) are universal arbitrary tails, e.g., suitable for PCR amplification.

### Amplification-Based Methods

In some embodiments, the KDR alteration among nucleic acid molecules isolated from the sample of the disclosure may be detected use an amplification-based method, wherein post amplification of KDR nucleic acids. As is known in the art, in such amplification-based methods, a sample of nucleic acids, such as a sample obtained from an individual or from a tumor, is used as a template in an amplification reaction (e.g., Polymerase Chain Reaction (PCR)) using one or more oligonucleotides or primers, e.g., such as one or more oligonucleotides or primers provided herein. The known nucleotide sequence for genes is sufficient to enable one of skill in the art to routinely select primers to amplify any portion of the gene. Fluorogenic quantitative PCR can also be used. In fluorogenic quantitative PCR, quantitation is based on the amount of fluorescence signals, e.g., TaqMan and Sybr green.

Other amplification methods suitable for use according to the methods provided herein include, e.g., ligase chain reaction (LCR), transcription amplification, self-sustained sequence replication, dot PCR, and linker adapter PCR.

In some aspects, provided herein are oligonucleotides, e.g., useful as primers. In some embodiments, an oligonucleotide, e.g., a primer, provided herein comprises a nucleotide sequence configured to hybridize to a target nucleic acid molecule comprising a KDR nucleic acid molecule provided herein, or a fragment or portion thereof. In some embodiments, the oligonucleotide comprises a nucleotide sequence configured to hybridize to the KDR nucleic acid molecule of the target nucleic acid molecule. In some embodiments, the oligonucleotides may be used for targeted sequencing as described herein.

In some embodiments, the oligonucleotide comprises a nucleotide sequence corresponding to a KDR nucleic acid molecule provided herein (e.g., a KDR nucleic acid molecule comprising a KDR alteration). In some embodiments, the oligonucleotide comprises a nucleotide sequence corresponding to a fragment or a portion of a KDR nucleic acid molecule provided herein. In some embodiments, the fragment or portion comprises between about 10 and about 30 nucleotides, between about 12 and about 20 nucleotides, or between about 12 and about 17 nucleotides. In some embodiments, the oligonucleotide comprises a nucleotide sequence complementary to a KDR nucleic acid molecule provided herein. In some embodiments, the oligonucleotide comprises a nucleotide sequence complementary to a fragment or a portion of a KDR nucleic acid molecule provided herein. In some embodiments, the fragment or portion comprises between about 10 and about 30 nucleotides, between about 12 and about 20 nucleotides, or between about 12 and about 17 nucleotides.

In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence, e.g., under high stringency conditions. In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence under conditions that allow a polymerization reaction (e.g., PCR) to occur.

In some embodiments, an oligonucleotide, e.g., a primer, provided herein may be useful for initiating DNA synthesis via PCR (polymerase chain reaction) or a sequencing method. In some embodiments, the oligonucleotide may be used to amplify a nucleic acid molecule comprising a KDR nucleic acid molecule provided herein, or a fragment thereof, e.g., using PCR. In some embodiments, the oligonucleotide may be used to sequence a nucleic acid molecule comprising a nucleic acid molecule comprising a KDR alteration as provided herein, or a fragment thereof.

In some embodiments, pairs of oligonucleotides, e.g., pairs of primers, are provided herein, which are configured to hybridize to a nucleic acid molecule comprising a KDR nucleic acid molecule provided herein, or a fragment thereof (e.g., a KDR nucleic acid molecule comprising a KDR alteration). In some embodiments, a pair of oligonucleotides of the disclosure may be used for directing amplification of the KDR nucleic acid molecule or fragment thereof, e.g., using a PCR reaction. In some embodiments, pairs of oligonucleotides, e.g., pairs of primers, are provided herein, which are configured to hybridize to a nucleic acid molecule comprising a KDR alteration provided herein, e.g., for use in directing amplification of the KDR nucleic acid molecule or fragment thereof, e.g., using a PCR reaction.

In some embodiments, an oligonucleotide, e.g., a primer, provided herein is a single stranded nucleic acid molecule, e.g., for use in sequencing or amplification methods described herein. In some embodiments, an oligonucleotide provided herein is a double stranded nucleic acid molecule. In some embodiments, a double stranded oligonucleotide is treated, e.g., denatured, to separate its two strands prior to use, e.g., in sequencing or amplification methods. Oligonucleotides provided herein comprise a nucleotide sequence of sufficient length to hybridize to their target, e.g., a KDR nucleic acid molecule provided herein, or a fragment thereof, and to prime the synthesis of extension products, e.g., during PCR or sequencing.

In some embodiments, an oligonucleotide, e.g., a primer, provided herein comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more deoxyribonucleotides or ribonucleotides. In some embodiments, an oligonucleotide provided herein comprises at least about 8 deoxyribonucleotides or ribonucleotides. In some embodiments, an oligonucleotide provided herein comprises at least about 10 deoxyribonucleotides or ribonucleotides. In some embodiments, an oligonucleotide provided herein comprises at least about 12 deoxyribonucleotides or ribonucleotides. In some embodiments, an oligonucleotide provided herein comprises at least about 15 deoxyribonucleotides or ribonucleotides. In some embodiments, an oligonucleotide provided herein comprises at least about 20 deoxyribonucleotides or ribonucleotides. In some embodiments, an oligonucleotide provided herein comprises at least about 30 deoxyribonucleotides or ribonucleotides. In some embodiments, an oligonucleotide provided herein comprises between about 10 and about 30 deoxyribonucleotides or ribonucleotides. In some embodiments, an oligonucleotide provided herein comprises between about 10 and about 25 deoxyribonucleotides or ribonucleotides. In some embodiments, an oligonucleotide provided herein comprises between about 10 and about 20 deoxyribonucleotides or ribonucleotides. In some embodiments, an oligonucleotide provided herein comprises between about 10 and about 15 deoxyribonucleotides or ribonucleotides. In some embodiments, an oligonucleotide provided herein comprises between about 12 and about 20 deoxyribonucleotides or ribonucleotides. In some embodiments, an oligonucleotide provided herein comprises between about 17 and about 20 deoxyribonucleotides or ribonucleotides. In some embodiments, the length and nucleotide sequence of an oligonucleotide provided herein is determined according to methods known in the art, e.g., based on factors such as the specific application (e.g., PCR, sequencing library preparation, sequencing), reaction conditions (e.g., buffers, temperature), and the nucleotide composition of the nucleotide sequence of the oligonucleotide or of its target complementary sequence.

In some embodiments, an oligonucleotide, e.g., a primer, of the disclosure distinguishes a nucleic acid, e.g., a genomic or transcribed nucleic acid, e.g., a cDNA or RNA, having a KDR alteration as described herein, from a reference nucleotide sequence.

### Sequencing

In some embodiments, a KDR alteration may be detected in a sample by sequencing, as described herein, such as by RNA sequencing or DNA sequencing. In some embodiments, DNA sequencing can be used to detect a KDR alteration. In some embodiments, RNA sequencing can be used to detect a KDR alteration. In some embodiments, the sequencing massively parallel sequencing (MPS) technique, whole genome sequencing (WGS), whole exome sequencing, targeted sequencing, direct sequencing, or a Sanger sequencing technique; an optionally wherein the massively parallel sequencing (MPS) technique comprises next-generation sequencing.

In some embodiments, a KDR alteration of the disclosure is detected using a sequencing method. Any method of sequencing known in the art can be used to detect a KDR alteration. Exemplary sequencing methods that may be used to detect a KDR alteration include those based on techniques developed by Maxam and Gilbert or Sanger. Automated sequencing procedures may also be used, e.g., including sequencing by mass spectrometry. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

In some embodiments, the sequencing may comprise the sequencing a massively parallel sequencing (MPS) technique, whole genome sequencing (WGS), whole exome sequencing, targeted sequencing, direct sequencing, or a Sanger sequencing technique; an optionally wherein the massively parallel sequencing (MPS) technique comprises next-generation sequencing. In some embodiments, the sequencing may be whole genome sequencing, whole exome sequencing, or targeted sequencing. In some embodiments, targeted sequencing may be used to sequence one or more subgenomic intervals. Targeted sequencing may be used to generate a plurality of sequencing reads overlapping one or more gene loci within one or more subgenomic intervals in a sample.

The disclosed methods and systems may be implemented using sequencing platforms such as the Roche/454 Genome Sequencer (GS) FLX System, Illumina/Solexa Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio® RS platform. In some instances, sequencing may comprise Illumina MiSeq™ sequencing. In some instances, sequencing may comprise Illumina HiSeq® sequencing. In some instances, sequencing may comprise Illumina NovaSeq® sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (c) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some embodiments, the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci. In some embodiments, the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof. In some embodiments, the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1B, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

In some embodiments, a KDR alteration nucleic acid molecules of the disclosure is detected using hybrid capture-based sequencing (hybrid capture-based NGS), e.g., using adaptor ligation-based libraries. Sec, e.g., Frampton, G. M. et al. (2013) Nat. Biotech. 31:1023-1031. In some embodiments, a KDR alteration of the disclosure is detected using next-generation sequencing (NGS). Next-generation sequencing includes any sequencing method that determines the nucleotide sequence of either individual nucleic acid molecules or clonally expanded proxies for individual nucleic acid molecules in a highly parallel fashion (e.g., greater than or equal to 105 molecules may be sequenced simultaneously). Next generation sequencing methods suitable for use according to the methods provided herein are known in the art and include, without limitation, massively parallel short-read sequencing, template-based sequencing, pyrosequencing, real-time sequencing comprising imaging the continuous incorporation of dye-labeling nucleotides during DNA synthesis, nanopore sequencing, sequencing by hybridization, nano-transistor array based sequencing, polony sequencing, scanning tunneling microscopy (STM)-based sequencing, or nanowire-molecule sensor based sequencing. See, e.g., Metzker, M. (2010) Nature Biotechnology Reviews 11:31-46, which is hereby incorporated by reference. Exemplary NGS methods and platforms that may be used to detect a [biomarker] nucleic acid molecule provided herein include, without limitation, the HeliScope Gene Sequencing system from Helicos BioSciences (Cambridge, MA., USA), the PacBio RS system from Pacific Biosciences (Menlo Park, CA, USA), massively parallel short-read sequencing such as the Solexa sequencer and other methods and platforms from Illumina Inc. (San Diego, CA, USA), 454 sequencing from 454 LifeSciences (Branford, CT, USA), Ion Torrent sequencing from ThermoFisher (Waltham, MA, USA), or the SOLID sequencer from Applied Biosystems (Foster City, CA, USA). Additional exemplary methods and platforms that may be used to detect a [biomarker] nucleic acid molecule provided herein include, without limitation, the Genome Sequencer (GS) FLX System from Roche (Basel, CHE), the G.007 polonator system, the Solexa Genome Analyzer, HiSeq 2500, HiSeq3000, HiSeq 4000, and NovaSeq 6000 platforms from Illumina Inc. (San Diego, CA, USA).

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147 (1): 195-197), the Striped Smith-Waterman algorithm (see, e.g., Farrar (2007), “Striped Smith-Waterman Speeds Database Searches Six Times Over Other SIMD Implementations”, Bioinformatics 23 (2): 156-161), the Needleman-Wunsch algorithm (Needleman, et al. (1970) “A General Method Applicable to the Search for Similarities in the Amino Acid Sequence of Two Proteins”, J. Molecular Biology 48 (3): 443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized.

In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26 (5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85 (6): 847-61. Examples of low-coverage SNP calling methods are described in, e.g., Li, Y., et al., Annu. Rev. Genomics Hum. Genet. 2009, 10:387-406.

After alignment, detection of substitutions and or other alterations can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜1e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates (e.g., non-frameshift insertion or non-frameshift deletions), and then these candidates are tested together with the reference sequence in a Bayesian framework.

Algorithms to generate candidate indels are described in, e.g., McKenna, A., et al., Genome Res. 2010; 20 (9): 1297-303; Ye, K., et al., Bioinformatics, 2009; 25 (21): 2865-71; Lunter, G., and Goodson, M., Genome Res. 2011; 21 (6): 936-9; and Li, H., et al. (2009), Bioinformatics 25 (16): 2078-9.

Methods for generating indel calls and individual-level genotype likelihoods include, e.g., the Dindel algorithm (Albers, C. A., et al., Genome Res. 2011; 21 (6): 961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21 (6): 952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix—Bioinformatics. 2010 Mar. 15; 26 (6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of exemplary nucleic acid sequencing methods, mutation calling methods, and methods for analysis of genetic variants is provided in, e.g., U.S. Pat. Nos. 9,340,830, 9,792,403, 11,136,619, 11,118,213, and International Patent Application Publication No. WO 2020/236941, the entire contents of each of which is incorporated herein by reference.

### Protein Detection Based Methods

In some embodiments, the KDR alteration from the sample of the disclosure may be detected using methods for detecting a KDR polypeptide.

In some embodiments, methods of detecting a KDR polypeptide comprise using any method known in the art, such as using antibodies (e.g., an antibody described herein), mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (R1A), immunofluorescent assays), and analytic biochemical methods (e.g., electrophoresis, capillary electrophoresis, high performance liquid chromatography (HPLC), thin layer chromatography (TLC), hyperdiffusion chromatography).

In some embodiments, antibodies or antibody fragments that specifically bind to a KDR polypeptide may be used to detect a KDR alteration. The antibody may be of any suitable type of antibody or antibody fragment, including, but not limited to, a monoclonal antibody, a polyclonal antibody, a multi-specific antibody (e.g., a bispecific antibody), or an antibody fragment, so long as the antibody or antibody fragment exhibits a specific antigen binding activity (e.g., binding to a KDR polypeptide of the disclosure, or a portion thereof). In some embodiments, the antibody may be a chimeric or humanized antibody. In certain embodiments, an antibody provided herein is a human antibody.

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some embodiments, a KDR alteration of the disclosure is detected in a sample comprising nucleic acids, e.g., genomic DNA, cDNA, or mRNA. In some embodiments, the sample is obtained from an individual having a cancer, such as a cancer described herein. A variety of materials (such as tissues) can be the source of the nucleic acid samples used in the methods provided herein. For example, the source of the sample can be solid tissue as from a fresh, frozen and/or preserved organ, tissue sample, biopsy, resection, smear, or aspirate; blood or any blood constituents; bodily fluids such as cerebrospinal fluid, amniotic fluid, urine, saliva, sputum, peritoneal fluid or interstitial fluid; or cells from any time in gestation or development of an individual. In some embodiments, the source of the sample is blood or blood constituents. In some embodiments, the source of the sample is a tumor sample. In some embodiments, the sample is or comprises biological tissue or fluid. In some embodiments, the sample can contain compounds that are not naturally intermixed with the tissue in nature, such as preservatives, anticoagulants, buffers, fixatives, nutrients, antibiotics or the like. In some embodiments, a KDR nucleic acid molecule is detected in a sample comprising genomic or subgenomic DNA fragments, or RNA, such as mRNA isolated from a sample, e.g., a tumor sample, a normal adjacent tissue (NAT) sample, a tissue sample, or a blood sample obtained from an individual. In some embodiments, the sample comprises cDNA derived from an mRNA sample or from a sample comprising mRNA. In some embodiments, the tissue is preserved as a frozen sample or as a formaldehyde- or paraformaldehyde-fixed paraffin-embedded (FFPE) tissue preparation. For example, the sample can be embedded in a matrix, e.g., an FFPE block or a frozen sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some embodiments, a sample is a primary sample obtained directly from a source of interest by any appropriate means. For example, in some embodiments, a primary biological sample is obtained by a method chosen from biopsy (e.g., fine needle aspiration or tissue biopsy), surgery, or collection of body fluid (e.g., blood, lymph, or feces). In some embodiments, as will be clear from context, the term “sample” refers to a preparation that is obtained by processing (e.g., by removing one or more components of and/or by adding one or more agents to) a primary sample. Such a processed sample may comprise, for example nucleic acids or proteins extracted from a sample or obtained by subjecting a primary sample to techniques such as amplification or reverse transcription of mRNA, or isolation and/or purification of certain components.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In one embodiment, the sample comprises one or more cells associated with a tumor, e.g., tumor cells or tumor-infiltrating lymphocytes (TIL). In one embodiment, the sample includes one or more premalignant or malignant cells. In one embodiment, the sample is acquired from a hematologic malignancy (or pre-malignancy), e.g., a hematologic malignancy (or pre-malignancy) described herein. In one embodiment, the sample is acquired from a cancer, such as a cancer described herein. In some embodiments, the sample is acquired from a solid tumor, a soft tissue tumor or a metastatic lesion. In other embodiments, the sample includes tissue or cells from a surgical margin. In another embodiment, the sample includes one or more circulating tumor cells (CTCs) (e.g., a CTC acquired from a blood sample). In one embodiment, the sample is a cell not associated with a tumor, e.g., a non-tumor cell or a peripheral blood lymphocyte.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some embodiments, the sample comprises tumor nucleic acids, such as nucleic acids from a tumor or a cancer sample, e.g., genomic DNA, RNA, or cDNA derived from RNA, from a tumor or cancer sample. In certain embodiments, a tumor nucleic acid sample is purified or isolated (e.g., it is removed from its natural state). In some embodiments, the sample is a control nucleic acid sample or a reference nucleic acid sample, e.g., genomic DNA, RNA, or cDNA derived from RNA, not containing a mutation or gene fusion described herein. In certain embodiments, the reference or control nucleic acid sample comprises a wild type or a non-mutated sequence. In certain embodiments, the reference nucleic acid sample is purified or isolated (e.g., it is removed from its natural state). In other embodiments, the reference nucleic acid sample is from a non-tumor sample, e.g., a blood control, a normal adjacent tumor (NAT), or any other non-cancerous sample from the same or a different subject.

In some instances, the sample comprises a polypeptide comprising a KDR alteration. The polypeptide may be detected from a solid tissue, e.g., from a fresh, frozen and/or preserved organ, tissue sample, biopsy (e.g., a tumor biopsy), resection, smear, or aspirate; blood or any blood constituents; bodily fluids such as cerebrospinal fluid, amniotic fluid, urine, saliva, sputum, peritoneal fluid or interstitial fluid; or cells such as tumor cells. In some embodiments, the source of the sample is blood or blood constituents. In some embodiments, the source of the sample is a tumor sample. In some embodiments, the sample is or comprises biological tissue or fluid. In some embodiments, the sample is preserved as a frozen sample or as a formaldehyde- or paraformaldehyde-fixed paraffin-embedded (FFPE) tissue preparation. In some embodiments, the sample comprises circulating tumor cells (CTCs). In some embodiments, the sample comprises tumor proteins or polypeptides, such as proteins or polypeptides from a tumor or a cancer sample. In certain embodiments, the proteins are purified or isolated (e.g., removed from their natural state).

In some instances, as noted above, the sample comprises nucleic acid or polypeptide (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some embodiments, the methods comprise detecting a KDR alteration in a sample from an individual. In some instances, the methods comprise obtaining knowledge of a KDR variant detected in a sample from a plurality of individuals with cancer. In some instances, the sample is obtained (e.g., collected) from an individual (e.g., patient) having or suspected of having cancer. In some instances, the cancer is a solid tumor or a metastatic form thereof. In some instances, the cancer is a hematological cancer, e.g., a leukemia or lymphoma. In some embodiments, the cancer is a vascular tumor such as but not limited to an angiosarcoma, cardiac sarcoma, hemangioma, or hemangioendothelioma, or a melanoma. In some embodiments, the cancer is breast angiosarcoma, heart sarcoma, kidney sarcoma, liver angiosarcoma, liver hemangioendothelioma, soft tissue angiosarcoma, skin sarcoma, soft tissue angiosarcoma, soft tissue hemangioendothelioma, soft tissue leiomyosarcoma, soft tissue sarcoma, unknown primary sarcoma, head and neck melanoma, skin melanoma, and unknown primary melanoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment)

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

The methods described herein relate to cancer ontologies (e.g., cancer types). In some embodiments, the cancers are from a preselected group of cancer ontologies. In some embodiments, the preselected group of cancer ontologies are chosen based on the association of the cancer with pathogenic KDR variants. In some embodiments, the preselected group of cancer ontologies comprise vascular tumors and melanoma.

In some embodiments, the preselected group of cancer ontologies comprise vascular tumors comprise angiosarcoma, cardiac sarcoma, hemangioma, and hemangioendothelioma.

In some embodiments, the preselected group of cancer ontologies comprise breast angiosarcoma, heart sarcoma, kidney sarcoma, liver angiosarcoma, liver hemangioendothelioma, soft tissue angiosarcoma, skin sarcoma, soft tissue angiosarcoma, soft tissue hemangioendothelioma, soft tissue leiomyosarcoma, soft tissue sarcoma, unknown primary sarcoma, head and neck melanoma, skin melanoma, and unknown primary melanoma.

In some embodiments, KDR variants in the preselected group of cancers are compared to KDR variants in any cancer ontology (e.g., cancer type). In some embodiments, knowledge of a KDR variant is obtained based on detection in samples from individuals with any cancer ontology including or not including cancer ontologies from the preselected group of cancer ontologies. In some embodiments, the cancer may be any one of acute myeloid leukemia, bile duct adenocarcinoma, bladder urothelial carcinoma, bone marrow lymphoproliferative disease, bone marrow multiple myeloma, bone marrow myeloproliferative neoplasm, brain anaplastic astrocytoma, brain glioblastoma, brain glioma, breast angiosarcoma, breast cancer, breast carcinoma, breast ductal carcinoma in situ, breast invasive ductal carcinoma, breast invasive lobular carcinoma, cervix adenocarcinoma, cervix squamous cell carcinoma, cholangiocarcinoma, colon adenocarcinoma, colon cancer, duodenum adenocarcinoma, esophagus adenocarcinoma, esophagus carcinoma, esophagus squamous cell carcinoma, eye intraocular melanoma, fallopian tube endometrioid carcinoma, gallbladder adenocarcinoma, gastroesophageal junction adenocarcinoma, head and neck carcinoma, head and neck melanoma, head and neck squamous cell carcinoma, heart sarcoma, kidney clear cell carcinoma, kidney collecting duct carcinoma, kidney renal cell carcinoma, kidney sarcoma, kidney urothelial carcinoma, liver angiosarcoma, liver cholangiocarcinoma, liver hemangioendothelioma, liver intrahepatic cholangiocarcinoma, lung adenocarcinoma, lung adenoid cystic carcinoma, lung cancer, lung large cell neuroendocrine carcinoma, lung non-small cell lung carcinoma, lung sarcomatoid carcinoma, lung small cell undifferentiated carcinoma, lung squamous cell carcinoma, lymph node leukemia lymphocytic acute, ovary endometrioid adenocarcinoma, ovary epithelial carcinoma, ovary germ cell tumor, ovary high grade serous carcinoma, ovary serous carcinoma, pancreas adenosquamous carcinoma, pancreas carcinoma, pancreas ductal adenocarcinoma, pancreas solid pseudopapillary tumor, pancreatobiliary carcinoma, pediatric soft tissue angiosarcoma, prostate acinar adenocarcinoma, prostate cancer, prostate ductal adenocarcinoma, rectum adenocarcinoma, salivary gland carcinoma, salivary gland duct carcinoma, skin basal cell carcinoma, skin melanoma, skin merkel cell carcinoma, skin sarcoma, skin squamous cell carcinoma, soft tissue angiosarcoma, soft tissue clear cell sarcoma, soft tissue ewing sarcoma, soft tissue fibromatosis, soft tissue hemangioendothelioma, soft tissue leiomyosarcoma, soft tissue sarcoma, soft tissue sarcoma undifferentiated, stomach adenocarcinoma, stomach adenocarcinoma diffuse type, stomach adenocarcinoma intestinal type, stomach carcinoma, thymus carcinoma, thyroid carcinoma, unknown primary adenocarcinoma, unknown primary cancer, unknown primary carcinoma, unknown primary f1h malignant neoplasm, unknown primary gist, unknown primary malignant neoplasm, unknown primary melanoma, unknown primary sarcoma, unknown primary squamous cell carcinoma, unknown primary urothelial carcinoma, ureter urothelial carcinoma, uterus endometrial adenocarcinoma, uterus endometrial adenocarcinoma endometrioid, uterus endometrial adenocarcinoma mixed histology, and vulva squamous cell carcinoma.

In some embodiments, a cancer of the disclosure, comprising a KDR variant, may be acute lymphoblastic leukemia (“ALL”), acute myeloid leukemia (“AML”), adenocarcinoma, adenocarcinoma of the lung, adrenocortical cancer, adrenocortical carcinoma, anal cancer, appendiceal cancer, B-cell derived leukemia, B-cell derived lymphoma, B-cell lymphoma, bladder cancer, brain cancer, breast cancer (e.g., triple negative breast cancer (TNBC) or non-triple negative breast cancer), cancer of the fallopian tube(s), cancer of the testes, carcinoma, cerebral cancer, cervical cancer, cholangiocarcinoma, choriocarcinoma, chronic myelogenous leukemia, central nervous system (CNS) tumor, CNS cancer, colon cancer, colorectal cancer (e.g., colon adenocarcinoma), diffuse intrinsic pontine glioma (DIPG), diffuse large B cell lymphoma (“DLBCL”), embryonal rhabdomyosarcoma (ERMS), endometrial cancer, epithelial cancer, epithelial neoplasm, thymoma, esophageal cancer, Ewing's sarcoma, eye cancer (e.g., uveal melanoma), eyelid cancer, follicular lymphoma (“FL”), gall bladder cancer, gastric cancer, gastrointestinal cancer, glioblastoma, polycythemia vera, glioblastoma multiforme, glioma (e.g., lower grade glioma), gullet cancer, head and neck cancer, a hematological cancer, hepatocellular cancer, hepatocellular carcinoma, Hodgkin's lymphoma (HL), a heavy chain disease, intestinum rectum cancer, renal cancer, kidney cancer (e.g., kidney clear cell cancer, kidney chromophobe cancer, kidney clear cell cancer, kidney papillary cancer), large B-cell lymphoma, large intestine cancer, laryngeal cancer, leucosis, leukemia, liver cancer, lung cancer (e.g., lung adenocarcinoma, or non-small cell lung cancer), lymphoma, mammary gland cancer, melanoma (e.g., metastatic malignant melanoma), Hodgkin's disease, Waldenstrom's macroglobulinemia, Merkel cell carcinoma, mesothelioma, monocytic leukemia, multiple myeloma, myeloma, myogenic sarcoma, nasopharyngeal cancer, neuroblastic-derived CNS tumor (e.g., neuroblastoma (NB)), neuroma, astrocytoma, pilocytic astrocytoma, anaplastic astrocytoma, medulloblastoma, craniopharyngioma, ependymoma, pincaloma, hemangioblastoma, acoustic neuroma, oligodendroglioma, meningioma, vestibular schwannoma, adenoma, metastatic brain tumor, spinal tumor, non-Hodgkin's lymphoma (NHL), oral cancer, oral cavity cancer, osteosarcoma, ovarian cancer, ovarian carcinoma, pancreatic adenocarcinoma, pancreatic cancer, peritoneal cancer, pheochromocytoma, primary mediastinal B-cell lymphoma, primary peritoneal cancer, prostate cancer (e.g., hormone refractory prostate adenocarcinoma), rectal cancer (rectum carcinoma), relapsed or refractory classic Hodgkin's Lymphoma (cHL), salivary gland cancer (e.g., salivary gland tumor), skin cancer, small cell lung cancer, small intestine cancer, soft tissue sarcoma, squamous carcinoma, squamous cell carcinoma (e.g., squamous cell carcinoma of the anogenital region, squamous cell carcinoma of the anus, squamous cell carcinoma of the cervix, squamous cell carcinoma of the esophagus, squamous cell carcinoma of the head and neck (SCHNC), squamous cell carcinoma of the lung, squamous cell carcinoma of the penis, squamous cell carcinoma of the vagina, or squamous cell carcinoma of the vulva), stomach cancer, T-cell derived leukemia, T-cell lymphoma, testicular cancer, testicular tumor, thymic cancer, thyroid cancer (thyroid carcinoma), tongue cancer, tunica conjunctiva cancer, urinary bladder cancer, urothelial cell carcinoma, uterine cancer (e.g., uterine endometrial cancer or uterine sarcoma such as uterine carcinosarcoma), uterine endometrial cancer, uterus cancer, vaginal cancer, vulvar cancer, or Wilms' tumor.

In some embodiments, a cancer of the disclosure, comprising a KDR variant, is a hematologic cancer (e.g., a hematologic malignancy), such as diffuse large B cell lymphoma (“DLBCL”), Hodgkin's lymphoma (“HL”), Non-Hodgkin's lymphoma (“NHL”), follicular lymphoma (“FL”), acute myeloid leukemia (“AML”), acute lymphoblastic leukemia (“ALL”), multiple myeloma (“MM”), acute lymphoblastic B-cell leukemia, acute lymphoblastic T-cell leukemia, acute myeloblastic leukemia, acute promyelocytic leukemia (“APL”), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (“CML”), chronic lymphocytic leukemia (“CLL”), or hairy cell leukemia. In some embodiments, a hematologic cancer of the disclosure, comprising a KDR variant, is an acute or a chronic leukemia, such as a lymphoblastic, myelogenous, lymphocytic, or myelocytic leukemia. In some embodiments, a hematologic cancer of the disclosure, e.g., comprising a KDR variant, is a lymphoma (e.g., Hodgkin's lymphoma, such as relapsed or refractory classic Hodgkin's Lymphoma (cHL), a non-Hodgkin's lymphoma, a diffuse large B-cell lymphoma, or a precursor T-lymphoblastic lymphoma), a lymphoepithelial carcinoma, or a malignant histiocytosis.

In some embodiments, a cancer of the disclosure, e.g., comprising a KDR variant, is a solid tumor (e.g., a solid malignancy), such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, osteosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilms tumor, cervical cancer, uterine cancer, testicular cancer, non-small cell lung cancer (NSCLC), small cell lung carcinoma, bladder carcinoma, lung cancer, epithelial carcinoma, skin cancer, melanoma, neuroblastoma (NB), or retinoblastoma.

In some embodiments, a cancer of the disclosure, e.g., comprising a KDR variant, is a, is a cancer of the adrenal glands (such as neuroblastoma), bladder cancer (such as urothelial (transitional cell) carcinoma), brain cancer (such as anaplastic astrocytoma or glioblastoma), bone cancer (such as osteosarcoma), bone marrow cancer (such as B-cell acute leukemia (B-ALL) or multiple myeloma), breast cancer (such as invasive ductal carcinoma), head and neck cancer (such as adenocarcinoma, mucoepidermoid carcinoma, squamous cell carcinoma), lymph node cancer, lung cancer (e.g., mucoepidermoid carcinoma, sarcoma, small cell undifferentiated carcinoma, adenocarcinoma, adenosquamous carcinoma, large cell carcinoma, large cell neuroendocrine carcinoma, non-small cell lung carcinoma, non-small cell lung carcinoma not otherwise specified, or squamous cell carcinoma), female reproductive cancer (e.g., cancer of the fallopian tubes such as fallopian tube serous carcinoma; ovarian cancer, such as epithelial carcinoma, epithelial carcinoma not otherwise specified, high grade serous carcinoma, low grade serous carcinoma, serous carcinoma; and uterine cancer, such as carcinosarcoma, endometrial adenocarcinoma, endometrial adenocarcinoma not otherwise specified, papillary serous endometrial adenocarcinoma, leiomyosarcoma, sarcoma, sarcoma not otherwise specified, or smooth muscle tumor of uncertain malignant potential (STUMP)), gallbladder cancer (such as adenocarcinoma), cancer of the gastroesophageal junction (such as adenocarcinoma), lymph node cancer (such as anaplastic large cell lymphoma, B-cell lymphoma, B-cell lymphoma not otherwise specified, diffuse large B cell lymphoma, non-Hodgkin's lymphoma, non-Hodgkin's lymphoma not otherwise specified), colon cancer (such as adenocarcinoma), colorectal cancer, skin cancer (such as melanoma or squamous cell carcinoma), small intestine cancer (adenocarcinoma), soft tissue cancer (such as Ewing sarcoma, fibrosarcoma, histiocytosis, histiocytosis not otherwise specified, juvenile xanthogranuloma or non-Langerhans cell histiocytosis, inflammatory myofibroblastic tumor, leiomyosarcoma, neurofibroma, neuroblastoma, sarcoma not otherwise specified, sarcoma, undifferentiated sarcoma, or an undifferentiated soft tissue cancer), pancreatic cancer (such as carcinoma, carcinoma not otherwise specified, ductal adenocarcinoma, or mucinous cystadenocarcinoma), prostate cancer (such as acinar adenocarcinoma), pericardium cancer (such as mesothelioma), peritoneum cancer (such as mesothelioma), salivary gland cancer (such as carcinoma or carcinoma not otherwise specified), stomach cancer (such as adenocarcinoma, adenocarcinoma not otherwise specified, or diffuse type cancer), kidney cancer (such as renal cell carcinoma or renal cell carcinoma not otherwise specified), thyroid cancer (such as carcinoma, carcinoma not otherwise specified, or papillary carcinoma), or a cancer of unknown primary origin (such as adenocarcinoma, carcinoma, carcinoma not otherwise specified, leiomyosarcoma, malignant neoplasm, malignant neoplasm not otherwise specified, melanoma, myoepithelial carcinoma, squamous cell carcinoma (SCC), or undifferentiated neuroendocrine carcinoma).

### Anti-Cancer Therapies

Certain aspects of the present disclosure relate to anti-cancer therapies. In some embodiments, an anti-cancer therapy of the disclosure includes one or more therapeutic agents, e.g., for treating a cancer.

In some embodiments, the anti-cancer therapy comprises a kinase inhibitor. In some embodiments, the methods provided herein comprise administering to the individual a kinase inhibitor, e.g., in combination with another anti-cancer therapy. In some embodiments, the kinase inhibitor is crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, or TAE684 (NVP-TAE684). Additional examples of ALK kinase inhibitors that may be used according to any of the methods provided herein are described in examples 3-39 of WO2005016894, which is incorporated herein by reference.

In some embodiments, the anti-cancer therapy is a kinase inhibitor. In some embodiments, the anti-cancer therapy is a kinase domain inhibitor. In some embodiments, the kinase domain inhibitor is selected from a groups consisting of sorafenib, sunitinib, cabozantinib, regorafenib, axitinib, apatinib, and ponatinib.

### EXEMPLARY EMBODIMENTS

The following exemplary embodiments are representative of some aspects of the invention:

Embodiment 1. A method of identifying an individual having an angiosarcoma who may benefit from a treatment comprising a kinase domain inhibitor, the method comprising detecting an alteration between amino acids 630 and 753 of kinase insert domain receptor (KDR) in a sample from the individual, wherein the presence of the alteration between amino acids 630 and 753 of KDR in the sample identifies the individual as on who may benefit from the treatment comprising the administration of a kinase domain inhibitor.

Embodiment 2. A method of selecting a therapy for an individual having cancer, the method comprising detecting an alteration between amino acids 630 and 753 of kinase insert domain receptor (KDR) in a sample from the individual, wherein the presence of the alteration between amino acid 630 and 753 of KDR in the sample identifies the individual as on who may benefit from treatment comprising the administration of a kinase domain inhibitor.

Embodiment 3. A method of identifying one or more treatment options for an individual having cancer, the method comprising: (a) detecting an alteration between amino acids 630 and 753 of kinase insert domain receptor (KDR) in a sample from the individual; and (b) generating a report comprising one or more treatment options identified for the individual based at least in part on the presence of the alteration between amino acid 630 and 753 of KDR in the sample wherein the one or more treatment options comprise the administration of a kinase domain inhibitor.

Embodiment 4. A method of treating or delaying progression of an angiosarcoma in an individual, comprising administering to the individual an effective amount of a kinase domain inhibitor, wherein the angiosarcoma comprises an alteration between amino acids 630 and 753 of the kinase insert domain receptor (KDR) gene.

Embodiment 5: The method of any of embodiments 1-4, wherein the alteration is between amino acids 665-743.

Embodiment 6: A method of identifying an individual having cancer who may benefit from a treatment comprising a kinase domain inhibitor, the method comprising: detecting an alteration in kinase insert domain receptor (KDR) in a sample from the individual; labeling the alteration as pathogenic if the alteration comprises a non-frameshift deletion affecting amino acid 630-753 of KDR and/or a non-frameshift insertion affecting amino acids 630-753 of KDR; wherein the labeling of the alteration as pathogenic identifies the individual as an individual who may benefit from treatment comprising the kinase domain inhibitor.

Embodiment 7: A method of treating or delaying progression of cancer in an individual having cancer, the method comprising: detecting an alteration in kinase insert domain receptor (KDR) in a sample from the individual; labeling the alteration as pathogenic if the alteration comprises a non-frameshift deletion affecting amino acid 630-753 of KDR and/or a non-frameshift insertion affecting amino acids 630-753 of KDR; administering to the individual a treatment comprising a kinase domain inhibitor if the alteration is labeled as pathogenic.

Embodiment 8: The method of embodiment 6 or 7, further comprising obtaining the sample from the individual.

Embodiment 9: The method of any of embodiments 6-8, wherein the kinase domain inhibitor is selected from a group consisting of KDR kinase inhibitors include sorafenib, sunitinib, cabozantinib, regorafenib, axitinib, apatinib, and ponatinib.

Embodiment 10: The method of any of embodiments 6-9, wherein the alterations labeled as pathogenic are any of the alterations of Table 2.

Embodiment 11: A method for classifying one or more regions of kinase insert domain receptor (KDR) as pathogenic in a preselected group of cancer ontologies, comprising: obtaining knowledge of a plurality of variants in KDR that have been detected in samples from a plurality of individuals having cancer; mapping the plurality of variants along the length of KDR by genomic location; identifying one or more regions of KDR with one or more variants from the plurality of variants associated with a cancer from the preselected group of cancer ontologies and without variants from the plurality of variants associated with cancers not in the preselected group of cancer ontologies; and classifying the identified one or more regions of KDR as pathogenic in the preselected group of cancer ontologies.

Embodiment 12: The method of any one of embodiments 6-11, wherein the cancer is a cancer from a preselected group of cancer ontologies.

Embodiment 13: The method of embodiment 11 or 12, wherein the preselected group of cancer ontologies comprise vascular tumors and melanoma.

Embodiment 14: The method of embodiment 13, wherein the vascular tumors comprise angiosarcoma, cardiac sarcoma, hemangioma, and hemangioendothelioma.

Embodiment 15: The method of embodiment 13 or 14, wherein the preselected group of cancer ontologies comprise breast angiosarcoma, heart sarcoma, kidney sarcoma, liver angiosarcoma, liver hemangioendothelioma, soft tissue angiosarcoma, skin sarcoma, soft tissue angiosarcoma, soft tissue hemangioendothelioma, soft tissue leiomyosarcoma, soft tissue sarcoma, unknown primary sarcoma, head and neck melanoma, skin melanoma, and unknown primary melanoma.

Embodiment 16: The method of any one of embodiments 11-15, wherein the one or more regions of KDR are within amino acid 630 to amino acid 753 of KDR.

Embodiment 17: The method of any one of embodiments 11-16, one or more regions of KDR comprise amino acid 665 to amino acid 743 of KDR.

Embodiment 18: The method of any of embodiments 11-17, wherein one or more selected variants in the one or more regions of KDR are classified as pathogenic.

Embodiment 19: The method of embodiment 18, wherein the one or more selected variant comprise any of the variants in Table 2.

Embodiment 20: The method of any one of embodiments 11-19, wherein the plurality of variants comprise non-frameshift alterations.

Embodiment 21: The method of embodiment 20, wherein the non-frameshift alterations comprise non-frameshift insertions, non-frameshift deletions, and/or substitutions.

Embodiment 22: The method of any one of embodiments 11-21, wherein obtaining knowledge of a plurality of variants in KDR that have been detected in samples from a plurality of individuals having cancer comprise detecting the plurality of variants in KDR in the samples.

Embodiment 23: The method of embodiment 22, wherein detecting the plurality of variants in KDR are detected in the samples by one or more methods selected from the group consisting of a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), and mass-spectrometric genotyping.

Embodiment 24: The method of any of embodiments 11-23, wherein the samples comprise tissue biopsy samples and/or liquid biopsy samples from the plurality of individuals having cancer.

Embodiment 25: The method of any one of embodiments 11-24, wherein the cancer any one of acute myeloid leukemia, bile duct adenocarcinoma, bladder urothelial carcinoma, bone marrow lymphoproliferative disease, bone marrow multiple myeloma, bone marrow myeloproliferative neoplasm, brain anaplastic astrocytoma, brain glioblastoma, brain glioma, breast angiosarcoma, breast cancer, breast carcinoma, breast ductal carcinoma in situ, breast invasive ductal carcinoma, breast invasive lobular carcinoma, cervix adenocarcinoma, cervix squamous cell carcinoma, cholangiocarcinoma, colon adenocarcinoma, colon cancer, duodenum adenocarcinoma, esophagus adenocarcinoma, esophagus carcinoma, esophagus squamous cell carcinoma, eye intraocular melanoma, fallopian tube endometrioid carcinoma, gallbladder adenocarcinoma, gastroesophageal junction adenocarcinoma, head and neck carcinoma, head and neck melanoma, head and neck squamous cell carcinoma, heart sarcoma, kidney clear cell carcinoma, kidney collecting duct carcinoma, kidney renal cell carcinoma, kidney sarcoma, kidney urothelial carcinoma, liver angiosarcoma, liver cholangiocarcinoma, liver hemangioendothelioma, liver intrahepatic cholangiocarcinoma, lung adenocarcinoma, lung adenoid cystic carcinoma, lung cancer, lung large cell neuroendocrine carcinoma, lung non-small cell lung carcinoma, lung sarcomatoid carcinoma, lung small cell undifferentiated carcinoma, lung squamous cell carcinoma, lymph node leukemia lymphocytic acute, ovary endometrioid adenocarcinoma, ovary epithelial carcinoma, ovary germ cell tumor, ovary high grade serous carcinoma, ovary serous carcinoma, pancreas adenosquamous carcinoma, pancreas carcinoma, pancreas ductal adenocarcinoma, pancreas solid pseudopapillary tumor, pancreatobiliary carcinoma, pediatric soft tissue angiosarcoma, prostate acinar adenocarcinoma, prostate cancer, prostate ductal adenocarcinoma, rectum adenocarcinoma, salivary gland carcinoma, salivary gland duct carcinoma, skin basal cell carcinoma, skin melanoma, skin merkel cell carcinoma, skin sarcoma, skin squamous cell carcinoma, soft tissue angiosarcoma, soft tissue clear cell sarcoma, soft tissue ewing sarcoma, soft tissue fibromatosis, soft tissue hemangioendothelioma, soft tissue leiomyosarcoma, soft tissue sarcoma, soft tissue sarcoma undifferentiated, stomach adenocarcinoma, stomach adenocarcinoma diffuse type, stomach adenocarcinoma intestinal type, stomach carcinoma, thymus carcinoma, thyroid carcinoma, unknown primary adenocarcinoma, unknown primary cancer, unknown primary carcinoma, unknown primary flh malignant neoplasm, unknown primary gist, unknown primary malignant neoplasm, unknown primary melanoma, unknown primary sarcoma, unknown primary squamous cell carcinoma, unknown primary urothelial carcinoma, ureter urothelial carcinoma, uterus endometrial adenocarcinoma, uterus endometrial adenocarcinoma endometrioid, uterus endometrial adenocarcinoma mixed histology, and vulva squamous cell carcinoma.

Embodiment 26: The method of any one of embodiments 6-10, wherein the method further comprises: providing a plurality of nucleic acid molecules obtained from the sample from the subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; and receiving, using one or more processors, sequence read data for the plurality of sequence reads, wherein the plurality of sequence reads comprise one or more sequence reads comprising an alteration in KDR.

Embodiment 27: The method of any one of embodiments 6-10 and 26, wherein the sample comprises a tissue biopsy sample, a liquid biopsy sample, or a normal control.

Embodiment 28: The method of embodiment 27, wherein the sample is a liquid biopsy sample and comprises blood, plasma, cerebrospinal fluid, sputum, stool, urine, or saliva.

Embodiment 29: The method of embodiment 28, wherein the sample is a liquid biopsy sample and comprises circulating tumor cells (CTCs).

Embodiment 30: The method of embodiment 28, wherein the sample is a liquid biopsy sample and comprises cell-free DNA (cfDNA).

Embodiment 31: The method of embodiment 30, wherein the cell-free DNA (cfDNA) or a portion thereof comprises circulating tumor DNA (ctDNA).

Embodiment 32: The method of any of embodiments 26-31, wherein the plurality of nucleic acid molecules comprises a mixture of tumor nucleic acid molecules and non-tumor nucleic acid molecules.

Embodiment 33: The method of embodiment 32, wherein the tumor nucleic acid molecules are derived from a tumor portion of a heterogeneous tissue biopsy sample, and the non-tumor nucleic acid molecules are derived from a normal portion of the heterogeneous tissue biopsy sample.

Embodiment 34: The method of embodiment 22, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

Embodiment 35: The method of any one of embodiments 26-34, wherein the one or more adapters comprise amplification primers, flow cell adaptor sequences, substrate adapter sequences, or sample index sequences.

Embodiment 36: The method of any one of embodiments 26-35, wherein the captured nucleic acid molecules are captured from the amplified nucleic acid molecules by hybridization to one or more bait molecules.

Embodiment 37: The method of embodiment 36, wherein the one or more bait molecules comprise one or more nucleic acid molecules, each comprising a region that is complementary to a region of a captured nucleic acid molecule.

Embodiment 38: The method of any one of embodiments 26-37, wherein amplifying nucleic acid molecules comprises performing a polymerase chain reaction (PCR) amplification technique, a non-PCR amplification technique, or an isothermal amplification technique.

Embodiment 39: The method of any one of embodiments 26-38, wherein the sequencing comprises use of a massively parallel sequencing (MPS) technique, whole genome sequencing (WGS), whole exome sequencing, targeted sequencing, direct sequencing, or Sanger sequencing technique.

Embodiment 40: The method of embodiment 39, wherein the sequencing comprises massively parallel sequencing, and the massively parallel sequencing technique comprises next generation sequencing (NGS).

Embodiment 41: The method of any one of embodiments 26-40, wherein the sequencer comprises a next generation sequencer.

Embodiment 42: The method of any one of embodiments 26-41, wherein one or more of the plurality of sequencing reads overlap one or more gene loci within one or more subgenomic intervals in the sample.

Embodiment 43: The method of embodiment 42, wherein the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

Embodiment 43: The method of embodiment 41 or 42, wherein the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.

Embodiment 44: The method of embodiment 41 or 42, wherein the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1B, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.

Embodiment 45: The method of any one of embodiments 6-10 and 26-44, further comprising generating, by the one or more processors, a report indicating the presence or absence of a KDR alteration labeled as pathogenic.

Embodiment 46: The method of embodiment 45, further comprising transmitting the report to a healthcare provider.

Embodiment 47: The method of embodiment 46, wherein the report is transmitted via a computer network or a peer-to-peer connection.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Liquid Biopsy-Based Diagnosis and Molecular Heterogeneity in a Child with Hepatic Angiosarcoma

This example provides a non-limiting example of the identification of a novel pathogenic gene alteration in the kinase insert domain receptor (KDR) gene between amino acids 630-753 (in exons 13-15) identified by a liquid biopsy sample from an angiosarcoma and the treatment of the angiosarcoma with a kinase domain inhibitor.

Present herein is an illustrative example of pediatric hepatic angiosarcoma where via ctDNA liquid biopsy comprehensive genomic profiling it was demonstrated that alterations in amino acids 630-753 of the KDR gene that were previously thought of as uncertain or benign are likely to be pathogenic driver alterations.

A 20-month old girl presented with progressive abdominal swelling. Blood tests indicated mild anemia (9.6 g/dL) and thrombocytopenia (104×10⁹/L). USG and CT abdomen revealed gross hepatomegaly as a result of multifocal hepatic lesions, with the dominant lesion measuring 13.9×7.5×10.1 cm, replacing most of segments 4, 7 and 8. These masses showed peripheral bizarre arterial enhancement with incomplete progressive fill-in of contrast, with irregular networks of dysplastic vessels, overall suggestive of hepatic vascular tumors. Consumptive hypothyroidism was present with markedly increased TSH (464 mIU/L, range: 0.7-6), low free T4 (2.5 pmol/L, range: 12-23), low free T3 (less than 1.5 pmol/L, range: 3.7-8.5), and increased reverse T3 (106 ng/dL, range: 8-25). Such endocrine dysfunction was most commonly present in patients with hemangioma, Simsek et al., Severe consumptive hypothyroidism caused by multiple infantile hepatic haemangiomas. 2018. J Pediatr Endocrinol Metab. 31 (7): 823-827, however, the patient age and radiographic features were atypical of such condition. With the inherent risk of bleeding associated with tumor biopsy, empirical treatment with vincristine and sirolimus was initiated.

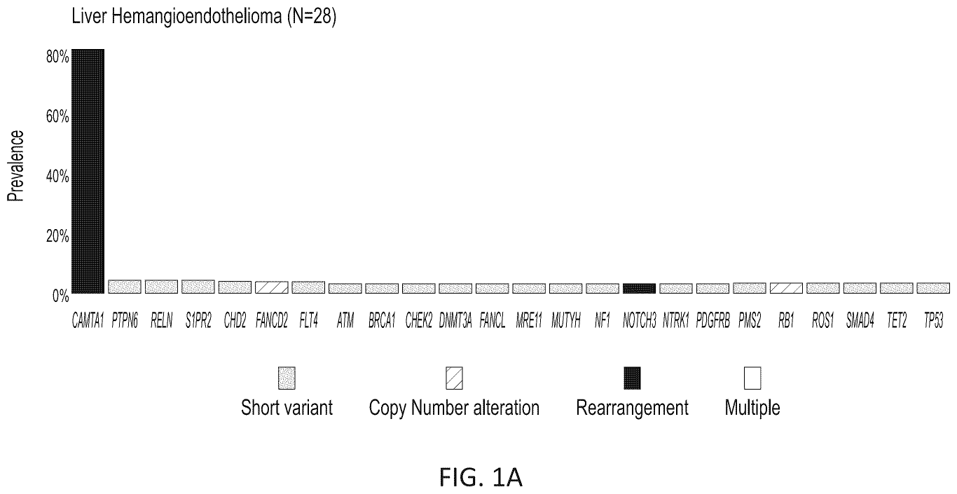

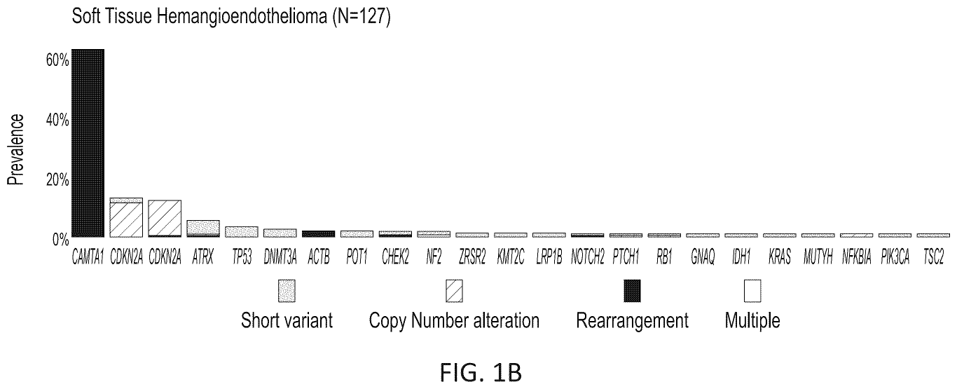

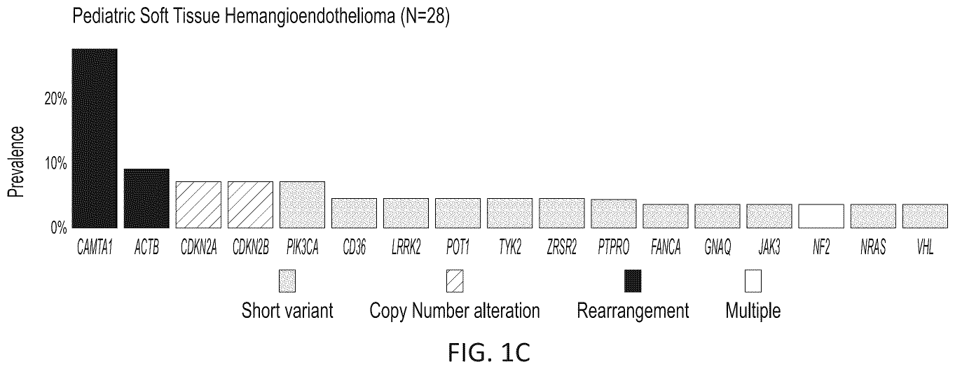

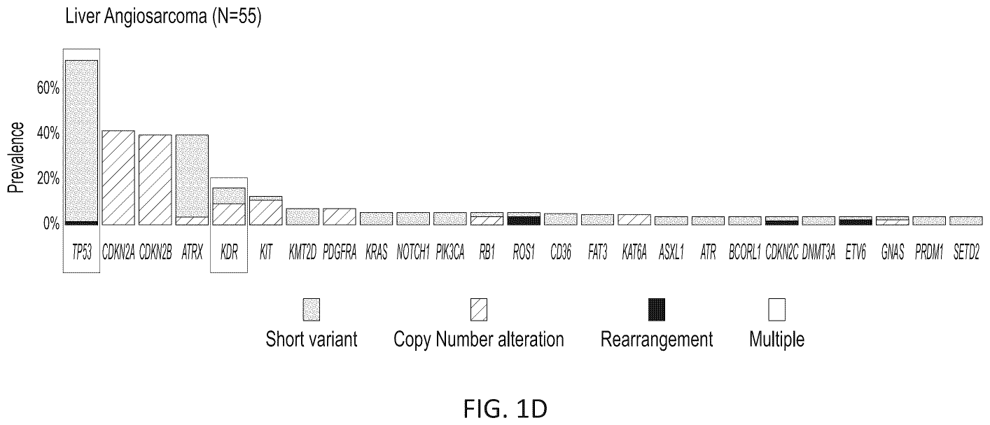

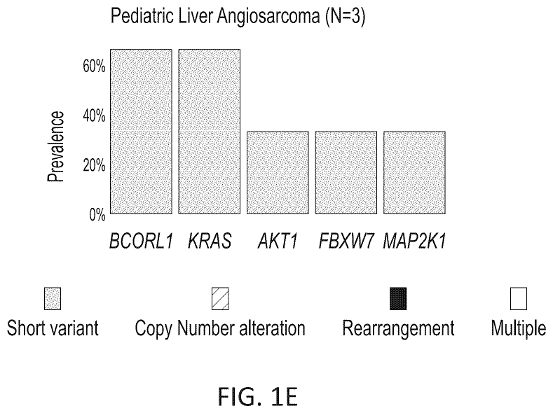

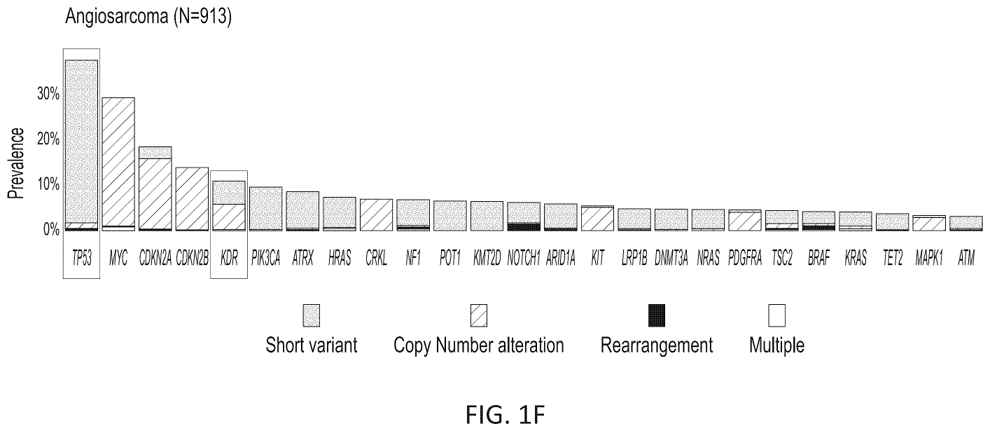

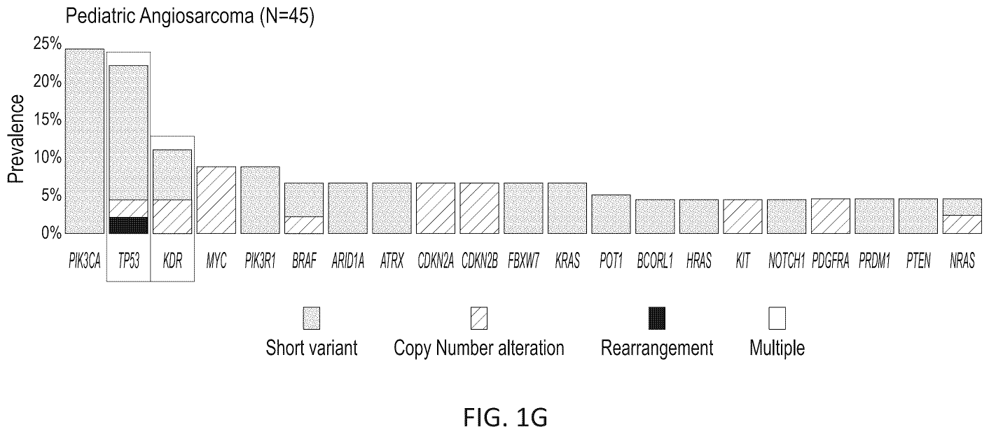

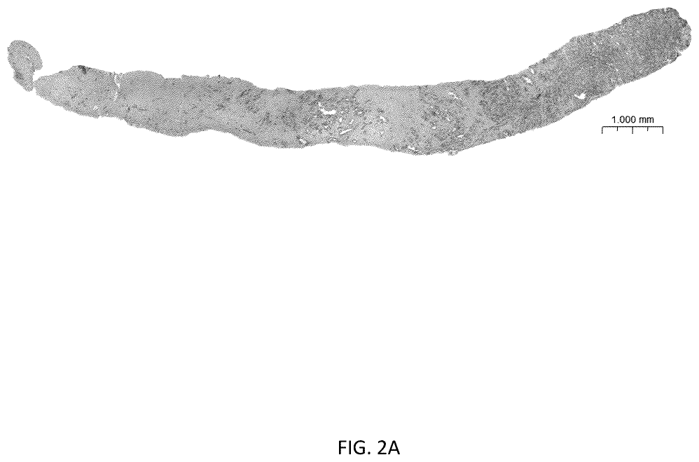

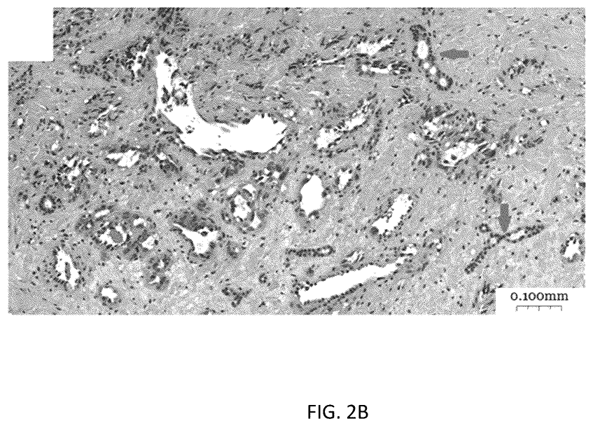

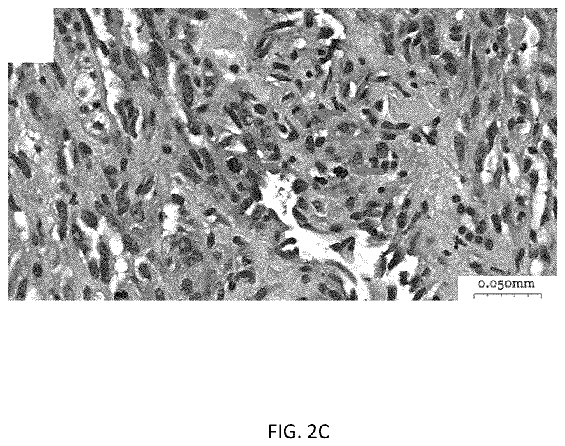

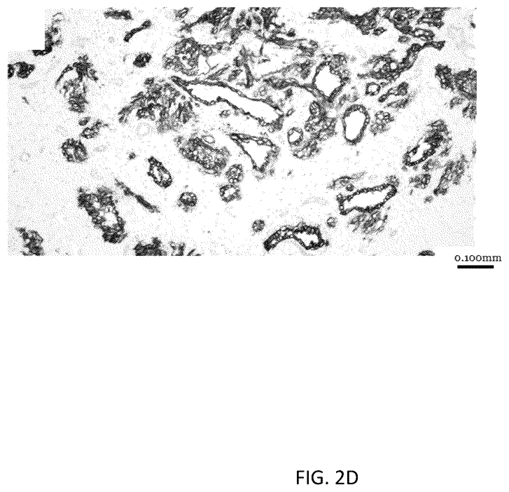

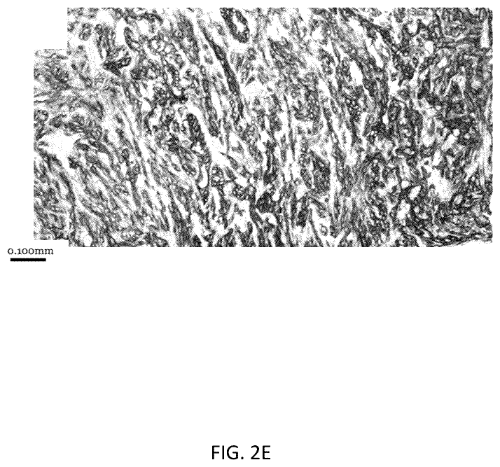

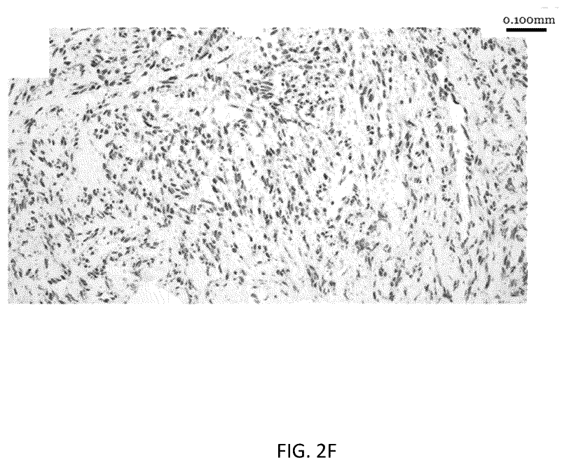

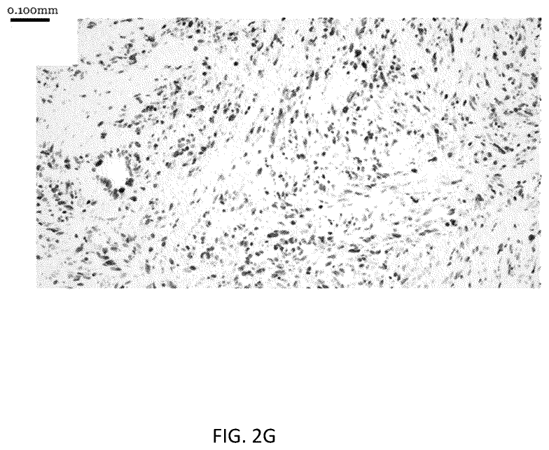

The patient displayed initial improvement with reduced hepatomegaly, normalization of blood counts and thyroid function, and radiographic evidence of tumor shrinkage (4 months from diagnosis). Ten weekly doses of vincristine were given after which, the patient was continued on sirolimus monotherapy. However, she developed abdominal distension again 10 months after the initial diagnosis. MRI confirmed significant progression of pre-existing bilobar multifocal lesions, with features concerning of angiosarcoma. With the concomitant ascites and coagulopathy, tumor biopsy was considered still to be hazardous, and instead, a plasma sample was sent for ctDNA tumor profiling. ctDNA tumor profiling unveiled known or likely pathogenic mutations in KDR (T771R VAF: 0.45%, D717_G718insLPLLGIVLKD VAF: 1.82%), BRAF (G469R VAF: 0.82%), TP53 (Q192*VAF: 0.26%, C242Y VAF: 0.22%, R273C VAF: 0.19%, G262D VAF: 0.11%, C135F VAF: 0.33%, N239_S240insN VAF: 0.71%), KRAS (D154del VAF: 44.4%), and BCORL1 (E1189fs*15 VAF: 46.8%) with a low ctDNA tumor fraction of less than 1%. As stated above, activating mutations in KDR, also known as VEGFR2, are known to drive angiosarcoma. Omiyale., Primary vascular tumours of the kidney. 2021. World J Clin Oncol. 12 (12): 1157-1168.; Antonescu, et al., KDR activating mutations in human angiosarcomas are sensitive to specific kinase inhibitors. 2009. Cancer Res, 69 (18): 7175-9. Referencing the genomic database (FIG. 1A-G and Table 1), mutations in KDR, TP53, BCOR, KRAS, and BRAF were commonly observed in angiosarcoma samples (n=913, but not hemangioma (n=28) or hemangioendothelioma (n=127), supporting the radiographic suspicion. KDR mutation were exclusively observed in angiosarcoma cases and TP53 alterations were frequent. (FIG. 1A-1G) Alterations were detected in either a tissue-based comprehensive genomic profiling assay, a hematologic comprehensive genomic profiling assay, and/or a blood-based comprehensive genomic profiling assay.

TABLE 1
KDR Exon 14/15 alterations observed in the genomic database, categorized
by Disease Ontology and adult/pediatric patient(s).

| | KDR alteration between | Additional KDR alterations | |
|---|---|---|---|
| Disease Ontology | amino acid 630-753 | identified | Adult/Pediatric |
| Soft tissue angiosarcoma | V665_P667>A | VUS: R720P | adult |
| Soft tissue angiosarcoma | V665_P667>A | | adult |
| Soft tissue angiosarcoma | T668_I669insT | VUS: S741C; D731E | adult |
| Soft tissue angiosarcoma | L673_E674insC | | adult |
| Breast angiosarcoma | E674_Q676del | Known/likely pathogenic: R725C | adult |
| Breast angiosarcoma | Q676_T677insENQ; S687_C688insFS | | adult |
| Breast invasive lobular carcinoma (ILC) | T678del | | adult |
| Lung non-small cell lung carcinoma | T678del | | adult |
| (NOS) | | | |
| Prostate cancer (NOS) | T678del | VUS: $1104Y | adult |
| Unknown primary cancer (NOS) | T678del | | adult |
| Breast angiosarcoma | I680_G681insGESI | | adult |
| Soft tissue angiosarcoma | E682del | | adult |
| Soft tissue angiosarcoma | E682_S683insS | VUS: D814L | adult |
| Soft tissue angiosarcoma | I684_E685insIGESI | | adult |
| Soft tissue angiosarcoma | I684_E685insGESI | | adult |
| Liver angiosarcoma | T678_A690del | VUS: C740R | adult |
| Breast angiosarcoma | N704del | | adult |
| Soft tissue angiosarcoma | N704del | | adult |
| Liver angiosarcoma | N704del | | adult |
| PEDIATRIC Soft tissue angiosarcoma | N704del | KDR amp CN 14 | pediatric |
| Soft tissue angiosarcoma | N704del | | adult |
| Soft tissue angiosarcoma | N704del | | adult |
| Head and neck melanoma | N704del | KDR amp CN 16 | adult |
| Unknown primary melanoma | N704del | VUS G1308R | adult |
| Soft tissue angiosarcoma | L715_K716insSIVL | | adult |
| Soft tissue angiosarcoma | R728_K729insR | | adult |
| Prostate acinar adenocarcinoma | R728_K729insR | VUS splice 1988-14_2037del64 | adult |
| Skin melanoma | R726_E730>K | | adult |
| Soft tissue angiosarcoma | D731_E732insLTIRRVRKED | | adult |
| Breast carcinoma (NOS) | D731_E732del | | adult |
| Breast angiosarcoma | R726_L734>S | | adult |
| Soft tissue angiosarcoma | L743_G744insCSVL | | adult |
| Breast angiosarcoma | L743del | VUS G681R | adult |

In the reported patient, a liquid biopsy performed using plasma-based cfDNA yielded more representative results of the tumor heterogeneity, capturing the key mutations of KDR and TP53. KDR encodes VEGFR2 and mediates VEGF-induced endothelial proliferation, survival, migration, tubular morphogenesis and sprouting. KDR T771R is a known activating hotspot mutation that has been implicated in the tumorigenesis of angiosarcoma, angioma and colorectal cancer, Antonescu, et al., KDR activating mutations in human angiosarcomas are sensitive to specific kinase inhibitors. 2009. Cancer Res, 69 (18): 7175-9; Lim, et al., Somatic p. T771R KDR (VEGFR2) mutation arising in a sporadic angioma during ramucirumab therapy. 2015. JAMA dermatology. 151 (11): 240-1243; Toledo, R. A., et al., Whole-exome sequencing of plasma cell-free DNA portrays the somatic mutation landscape of refractory metastatic colorectal cancer and enables the discovery of mutated KDR/VEGFR2 receptors as modulators of anti-angiogenic therapies. 2017. bioRxiv. 177287, increasing protein signaling through dimerization and stabilization of protein.

The second KDR alteration identified in the ctDNA specimen the patient, D717_G718insLPLLGIVLKD, was initially regarded as a variant of uncertain significance. Upon review of a genomic database, an enrichment of angiosarcoma cases with nucleotide insertions or deletions at similar positions was observed (Table 1). Further, for the pediatric age group, Whitlock et al. also reported a novel activating internal tandem duplication in the KDR gene in a case with pancreatic angiosarcoma in the same region as the internal tandem duplication identified in the initial patient. Whitlock, R. S., et al., Angiosarcoma of the Pancreas in a Pediatric Patient With an Activating KDR-Internal Tandem Duplication: A Case Report and Review of the Literature. 2022. J Pediatr Hematol Oncol, 2022. 44 (3): p. e751-e755. Taken together, the KDR VUS identified in the patient's case and similar KDR VUS's were reclassified as likely pathogenic.

Via the use of ctDNA profiling, it was possible to reassign the status of D717_G718insLPLLGIVLKD by identifying a novel region of the gene that is likely to impact oncogenicity. As a result, it was possible to diagnose and prompt the adoption of a kinase domain inhibitor like a pazopanib-based regimen, which resulted in the diagnosis and prompted the adoption of a pazopanib-based regimen, dramatic tumor response. In particular, it was discovered that non-frameshift insertions and deletions impacting, but not required to be entirely located within amino acids 630-753 of the KDR transcript NM_002253.4 play a role in the pathogenicity of angiosarcomas, cardiac sarcomas, and melanoma and thus determining the associated status of the variant.

### Example 2: Classification of KDR Region as Pathogenic in Vascular Tumors and Melanoma

This example provides a non-limiting example of classification of regions of the KDR gene as pathogenic in a group of cancer ontologies.

A list of KDR non-frameshift variants that were detected in either a tissue-based comprehensive genomic profiling assay, a hematologic comprehensive genomic profiling assay, and/or a blood-based comprehensive genomic profiling assay was acquired.

A list of cancer ontologies were selected that were expected to exhibit pathogenic KDR mutations. Angiosarcoma, cardiac sarcoma, hemangioma, and hemangioendothelioma were chosen because they comprise vascular tumors, and KDR mutations are known to be pathogenic in angiosarcoma. Melanoma was also chosen because KDR mutations are known to be recurrent in this context (Hodis et al., 2012; U.S. Pat. No. 22,817,889, Krauthammer et al., 2012; U.S. Pat. No. 22,842,228, Xia et al., 2014; 24755198). The list of selected cancer ontologies included, breast angiosarcoma, heart sarcoma, kidney sarcoma, liver angiosarcoma, liver hemangioendothelioma, soft tissue angiosarcoma, skin sarcoma, soft tissue angiosarcoma, soft tissue hemangioendothelioma, soft tissue leiomyosarcoma, soft tissue sarcoma, unknown primary sarcoma, head and neck melanoma, skin melanoma, and unknown primary melanoma.

The individual KDR variants were sorted according to type (non-frameshift insertion, non-frameshift deletion, non-frameshift indel) and the cancer ontology the sample was annotated as. The variants were mapped by genomic position. (FIG. 3A, 3B, 4A, 4B, 5A, 5B) Regions were identified by visual inspection of the observation of variants within the selected cancer ontologies that were chosen based on their association with pathogenic KDR variants. Enrichment of non-frameshift KDR mutations in the selected cancer ontologies comped to all tumor types was identified between amino acid 630 to amino acid 753 of KDR. The non-frameshift mutations between amino acid 630 and amino acid 753 of KDR were thus classified as pathogenic in the selected cancer ontologies.

Table 2 provides the identified KDR alteration between amino acid 630 and amino acid 753 of the KDR gene in samples from patients with a cancer from preselected cancer ontologies. Table 2 provides an expanded group of detected variants compared to those reported in Table 1.

TABLE 2
Exemplary identified variants in the identified KDR region

| Disease Ontology | KDR alteration between amino acid 630-753 |
|---|---|
| Heart sarcoma (NOS) | K630_A644del |
| Soft tissue angiosarcoma | N622_T649del |
| Breast angiosarcoma | P667del |
| Soft tissue angiosarcoma | V665_P667>A |
| Heart sarcoma (NOS) | P667del |
| Soft tissue sarcoma (NOS) | V665_P667del |
| Soft tissue angiosarcoma | T668_I669insT |
| Soft tissue angiosarcoma | A666_I669>V |
| Breast angiosarcoma | G671_L673>C |
| Soft tissue angiosarcoma | L673_E674insC |
| Breast angiosarcoma | E674_Q676del |
| Soft tissue sarcoma (NOS) | N675_Q676del |
| Soft tissue sarcoma (NOS) | T678_S679insT |
| Breast angiosarcoma | I680_G681insGESI |
| Soft tissue angiosarcoma | I680_G681insACI |
| Soft tissue angiosarcoma | E682del |
| Soft tissue angiosarcoma | E682_S683insS |
| Heart sarcoma (NOS) | S683_I684insKS |
| Soft tissue angiosarcoma | I684_E685insIGESI |
| Soft tissue hemangioendothelioma | I684_E685insGESI |
| Soft tissue angiosarcoma | I680_C688del |
| Soft tissue angiosarcoma | C688_T689insC |
| Soft tissue angiosarcoma | E685_C688del |
| Unknown primary sarcoma (NOS) | E682_C688del |
| Heart sarcoma (NOS) | C688_T689insSIGESIEVSC |
| Unknown primary sarcoma (NOS) | S683_C688del |
| Soft tissue angiosarcoma | C688_T689del |
| Soft tissue angiosarcoma | T689_A690insCT |
| Soft tissue angiosarcoma | E685_T689del |
| Liver angiosarcoma | T678_A690del |
| Soft tissue angiosarcoma | V665_F701del |
| Skin sarcoma (NOS) | F701_K702ins24 |
| Soft tissue angiosarcoma | F701_K702ins24 |
| Breast angiosarcoma | N704del |
| Soft tissue angiosarcoma | N704del |
| Heart sarcoma (NOS) | N704del |
| Liver angiosarcoma | N704del |
| PEDIATRIC Soft tissue angiosarcoma | N704del |
| Soft tissue angiosarcoma | N704del |
| Breast angiosarcoma | L715_K716insLGIVL |
| Soft tissue angiosarcoma | L715_K716insSIVL |
| Liver hemangioendothelioma | D717_G718insLPLLGIVLKD |
| Heart sarcoma (NOS) | L722_T723insI |
| Soft tissue hemangioendothelioma | V727_R728insV |
| Soft tissue angiosarcoma | R728_K729insR |
| Breast angiosarcoma | R728_K729insNLTIRRVR |
| Heart sarcoma (NOS) | E730_D731insE |
| Soft tissue angiosarcoma | D731_E732insLTIRRVRKED |
| Heart sarcoma (NOS) | E732_G733insDE |
| Breast angiosarcoma | R726_L734>S |
| Soft tissue angiosarcoma | L743_G744insCSVL |
| Breast angiosarcoma | L743del |
| Heart sarcoma (NOS) | C745_E749>W |

What is claimed is:

1. A method of identifying an individual having cancer who may benefit from a treatment comprising a kinase domain inhibitor, the method comprising:
detecting an alteration in kinase insert domain receptor (KDR) in a sample from the individual;
labeling the alteration as pathogenic if the alteration comprises a non-frameshift deletion affecting amino acid 630-753 of KDR and/or a non-frameshift insertion affecting amino acids 630-753 of KDR;
wherein the labeling of the alteration as pathogenic identifies the individual as an individual who may benefit from treatment comprising the kinase domain inhibitor.

2. A method of treating or delaying progression of cancer in an individual having cancer, the method comprising:
detecting an alteration in kinase insert domain receptor (KDR) in a sample from the individual;
labeling the alteration as pathogenic if the alteration comprises a non-frameshift deletion affecting amino acid 630-753 of KDR and/or a non-frameshift insertion affecting amino acids 630-753 of KDR;
administering to the individual a treatment comprising a kinase domain inhibitor if the alteration is labeled as pathogenic.

3. The method of claim 1, further comprising obtaining the sample from the individual.

4. The method of claim 1, wherein the kinase domain inhibitor is selected from a group consisting of KDR kinase inhibitors include sorafenib, sunitinib, cabozantinib, regorafenib, axitinib, apatinib, and ponatinib.

5. The method of claim 1, wherein the alterations labeled as pathogenic are any of the alterations of Table 2.

6. The method of claim 1, wherein the cancer is a cancer from a preselected group of cancer ontologies.

7. The method of claim 6, wherein the preselected group of cancer ontologies comprise vascular tumors and melanoma.

8. The method of claim 6, wherein the preselected group of cancer ontologies comprise breast angiosarcoma, heart sarcoma, kidney sarcoma, liver angiosarcoma, liver hemangioendothelioma, soft tissue angiosarcoma, skin sarcoma, soft tissue angiosarcoma, soft tissue hemangioendothelioma, soft tissue leiomyosarcoma, soft tissue sarcoma, unknown primary sarcoma, head and neck melanoma, skin melanoma, and unknown primary melanoma.

9. A method for classifying one or more regions of kinase insert domain receptor (KDR) as pathogenic in a preselected group of cancer ontologies, comprising:
obtaining knowledge of a plurality of variants in KDR that have been detected in samples from a plurality of individuals having cancer;
mapping the plurality of variants along the length of KDR by genomic location;
identifying one or more regions of KDR with one or more variants from the plurality of variants associated with a cancer from the preselected group of cancer ontologies and without variants from the plurality of variants associated with cancers not in the preselected group of cancer ontologies; and
classifying the identified one or more regions of KDR as pathogenic in the preselected group of cancer ontologies.

10. The method of claim 9, wherein the cancer is a cancer from a preselected group of cancer ontologies.

11. The method of claim 9, wherein the preselected group of cancer ontologies comprise vascular tumors and melanoma.

12. The method of claim 11, wherein the vascular tumors comprise angiosarcoma, cardiac sarcoma, hemangioma, and hemangioendothelioma.

13. The method of claim 11, wherein the preselected group of cancer ontologies comprise breast angiosarcoma, heart sarcoma, kidney sarcoma, liver angiosarcoma, liver hemangioendothelioma, soft tissue angiosarcoma, skin sarcoma, soft tissue angiosarcoma, soft tissue hemangioendothelioma, soft tissue leiomyosarcoma, soft tissue sarcoma, unknown primary sarcoma, head and neck melanoma, skin melanoma, and unknown primary melanoma.

14. The method of claim 9, wherein the one or more regions of KDR are within amino acid 630 to amino acid 753 of KDR.

15. The method of claim 9, one or more regions of KDR comprise amino acid 665 to amino acid 743 of KDR.

16. The method of claim 9, wherein one or more selected variants in the one or more regions of KDR are classified as pathogenic.

17. The method of claim 16, wherein the one or more selected variant comprise any of the variants in Table 2.

18. The method of claim 8, wherein the plurality of variants comprise non-frameshift alterations.

19. The method of claim 18, wherein the non-frameshift alterations comprise non-frameshift insertions, non-frameshift deletions, and/or substitutions.

20. The method of claim 9, wherein obtaining knowledge of a plurality of variants in KDR that have been detected in samples from a plurality of individuals having cancer comprise detecting the plurality of variants in KDR in the samples.
